(19) United States
(12) Patent Application Publication
(10) Pub. No.: US 2015/0351707 A1
(43) Pub. Date: Dec. 10, 2015

(54) RADIAL ACCESS PROCEDURES AND DEVICES

(71) Applicant: G2 MEDICAL, LLC, MONROE, LA (US)

(72) Inventor: Gregory SAMPOGNARO, Monroe, LA (US)

(73) Assignee: G2 MEDICAL, LLC, MONROE, LA (US)

(21) Appl. No.: 14/327,336

(22) Filed: Jul. 9, 2014

Related U.S. Application Data
(60) Provisional application No. 62/008,321, filed on Jun. 5, 2014.

(51) Int. Cl. A61B 6/04; A61M 5/52; A61G 13/12

(52) U.S. Cl. CPC A61B 6/0428; A61G 13/1205; A61G 13/1235; A61M 5/52

(57) ABSTRACT

Radial access methods and apparatus are described for facilitating the securement of the patient's limb for a radial procedure as well as to accommodate for the re-positioning of the patient's body. A radial table assembly may generally comprise a platform defining a surface which is sized to support the limb of the patient, an interface portion attached at a distal end of the platform, wherein the interface portion is radio-lucent and extends at a distance from the platform, and a cradle member rotatably connected to the platform at a location proximal to the interface portion. In other variations, the interface portion is reconfigurable relative to the platform such that an angled edge is reversible relative to the platform.

### CROSS-REFERENCE TO RELATED APPLICATIONS

This application claims the benefit of priority to U.S. Prov. App. 62/008,321 filed Jun. 5, 2014, which is incorporated herein by reference in its entirety.

### FIELD OF THE INVENTION

The present invention relates to methods and apparatus for facilitating radial access procedures. More particularly, the present invention relates to methods and apparatus for facilitating radial access procedures when repositioning the patient is needed or desired.

### BACKGROUND OF THE INVENTION

Cardiac catheterization procedures generally utilize a catheter passed through a major artery, such as a femoral artery, to access a patient's heart. Yet access to the intravascular and cardiac systems may also be achieved through a radial artery and is generally believed to be more convenient and may also reduce both the length of the procedure and the risk of complications during the procedure. However, cardiac catheterization procedures often require the use of numerous surgical instruments including guidewires, intravascular balloons, stents, etc.

Moreover, imaging systems such as fluoroscopes or other imaging machines are typically utilized during catheterization procedures. Due to the size and weight of these machines, imaging various tissue regions within the patient's body often necessitates re-positioning of the body relative to the imaging system. This requires physically moving the patient's body. However, because of the number of instruments used during a radial access procedure, re-positioning the patient's body is difficult and cumbersome.

Previous devices have been developed to facilitate radial access. Such devices have included the use of boards or surfaces which extend from the platform upon which the patient is positioned. Such boards are typically clamped or otherwise secured to the platform for supporting the patient's limb, such as their arm, to extend from the body to provide radial access. However, such boards are cumbersome to use in a surgical setting and fail to provide a stable platform upon which instruments can be placed.

Other devices have included the use of arm boards or platforms supported by pivoting or jointed arms which can be clamped upon the catheter laboratory table or a separate supporting structure. While such pivoted or jointed arms are able to support a patient's arm for a radial procedure, these devices still fail to provide any stable platform for instruments and further fail to address the need for moving or re-positioning the patient's body relative to an imaging system.

Accordingly, there exists a need for methods and devices which are able to support any number of instruments for radial access as well as facilitate re-positioning of the patient's body during a radial access procedure.

### SUMMARY OF THE INVENTION

In accessing a radial vessel, e.g., for an interventional procedure, the limb of the patient, such as an arm, is generally positioned to extend the wrist and expose a radial artery. To facilitate the securement of the patient's limb for a radial procedure as well as to accommodate for the re-positioning of the patient's body, a radial table assembly as described may be utilized. The table assembly may generally comprise a platform defining a surface which is sized to support the limb of the patient, an interface portion attached at a distal end of the platform, wherein the interface portion is radio-lucent and extends at a distance from the platform, and a cradle member rotatably connected to the platform at a location proximal to the interface portion. In other variations, the interface portion is reconfigurable relative to the platform such that an angled edge is reversible relative to the platform.

The cradle assembly itself may be generally comprised of a cradle member which is configured to receive and support the limb of the patient, a connection interface configured to rotatably secure the assembly to a first platform, and a support arm rotatably extending from the connection interface and rotatably coupled to the cradle member, wherein a position and an orientation of the cradle member is dependent upon a position and an orientation of a second platform.

In use, one method for positioning the patient body with respect to the radial table assembly may generally comprise positioning the limb of the patient body upon the cradle member which is movably attached to a first platform sized to support the limb, moving a second platform upon which the patient body is placed, where the second platform is in proximity to the first platform, and moving the cradle member relative to the first platform in a manner corresponding to a movement of the second platform such that a position or orientation of the cradle member is dependent upon a position or orientation of the second platform when the limb is positioned upon the cradle member.

One example of a radial table assembly may generally comprising a back table supported by one or more legs, a radial table portion extending from the back table, and an interface portion further extending from the radial table portion, e.g., in a cantilevered manner. The interface portion may define an angled edge which allows for the table assembly to be positioned relatively at an angle and in close proximity to or adjacent to a catheter laboratory table and provide a platform upon which the patient's limb (such as the arm) may be extended from the patient body positioned upon the table. The table assembly may provide surgeons and practitioners a comfortable way to facilitate procedures, e.g., angiography, percutaneous interventions, etc., through the radial artery.

The back table may comprise a surgical table or platform and the radial table portion may be attached to a distal edge of the back table along an optionally hinged or pivoting interface and having a width of, e.g., 12-48 inches or more, and a length of, e.g., up to 20 inches or longer. The interface portion may be secured at a distal end of the radial table portion also along an optionally hinged or pivoting interface and may have a similar width as the radial table portion. The interface portion may extend at a distance and terminate at its distal end at the angled edge which may have an angle ranging anywhere from, e.g., 10 degrees and up to 90 degrees (or more particularly 45-55 degrees), relative to the length of the table assembly. While each of the back table and radial table portion may be comprised of various materials which are radio-opaque, e.g., stainless steel, the interface portion may be comprised of a radio-lucent material which is optionally anti-bacterial or able to be disinfected, e.g., various polycarbonate materials such as Lexan® (SABIC Innovative Plastics), ultra-high-molecular-weight polyethylene (UHMW), etc. Having the interface portion comprised of a radio-lucent material may allow for an imaging device such as a fluoroscope (or any other suitable imaging device) to penetrate and image the proximal upper sections of the patient's limb, if needed.

A cradle assembly may be rotatably secured to the radial table assembly within any one of the connection interfaces which may be aligned along, e.g., the proximal edge of the radial table portion. The cradle assembly may be optionally positioned in any of the connection interfaces depending upon the positioning of the patient body along the catheter laboratory table. The cradle assembly itself may generally comprise a cradle member having a length of, e.g., up to 10 inches or more, and a width of, e.g., 6 inches or more, and which is configured to define a channel to comfortably receive the patient's wrist, forearm, and hand. An optional proximal cradle member may extend adjustably via an extension arm from the cradle member to support the patient's upper arm if needed or desired. The cradle member and optional proximal cradle member may each be fabricated from either a radio-opaque material (such as stainless steel) or from a radio-lucent material (such as polycarbonate) to further allow for imaging of the forearm and hand of the patient if also desired.

The cradle assembly may define two points of rotation or articulation to enable the rotation of the cradle member relative to a support arm which couples the cradle assembly to the connection interface and to also enable the rotation of the support arm relative to the connection interface and radial table. With the patient body positioned upon the catheter laboratory table, the limb and particularly the hand and wrist may be extended and secured upon a cradle member with the patient's fingers optionally secured to the distal end of the cradle member to extend the wrist. With the limb secured and optionally draped, the radial artery may be readily accessed by any number of percutaneous access or surgical procedures. The surgeon and/or practitioner may be positioned between the catheter laboratory or operating table and the radial table assembly while any number of instruments or interventional tools may be placed or extended upon the radial table portion and/or back table.

However, if the patient body requires repositioning relative to the imaging device positioned above and below the patient and laboratory table, the laboratory table may be translated either along its length or width while keeping the limb secured to the cradle member. Hence, as the patient body moves relative to the imaging device and relative to the radial table assembly, the patient's limb may also be repositioned in a manner corresponding to the repositioning of the patient body due to the rotation of cradle member (and the hand and wrist positioned upon the cradle member) relative to the support arm and also the rotation of the support arm relative to the radial table portion without interference with the surgeon or practitioner. These two points of rotation of the cradle member and the support arm relative to the radial table may allow for a full 360 degrees or movement. Alternatively, the rotation of one or both of the cradle member and support arm relative to the radial table portion may be limited to rotate within specified ranges, if so desired. In either case, because the orientation of the patient's limb may be automatically maintained relative to the patient's body despite any translation of the body relative to the imaging device and/or radial table assembly, any instruments or tools extending from the patient's limb and laid upon the back table may be left in place rather than moved to accommodate movement of the patient body.

Because the radial table assembly may be utilized with a number of different imaging devices, the assembly may be designed to accommodate an imaging system such as a fluoroscope. Such systems typically utilize a C-arm having a detector and x-ray transmitter positioned in apposition to one another at either end of the C-arm. While the imaging system is generally stationary relative to the laboratory table and radial table assembly, the C-arm may be rotatably adjustable for obtaining images of the underlying patient body at various angles. To accommodate such movement of the imaging system, the radial table assembly may be configured to have the radial table portion and interface portion extend at a distance, e.g., anywhere from a foot or up to several feet (or more particularly between 5-6 feet), from the back table in a cantilevered manner to provide for sufficient clearance distance beneath the table in the event that the transmitter (or any other instrument) is positioned beneath the assembly. This cantilevered feature may be incorporated into any of the various embodiments of the table assembly shown or described herein. Measurements and dimensions with respect to the radial table assembly are provided as illustrative examples and may be varied or customized to suit any number of patients as desired.

Because the interface portion defines an angled edge, the interface portion may be adjustably attached to the radial table portion. Yet another feature may include the radial table portion being retractably folded along its interface relative to the back table. Other variations of the radial table may include embodiments where the radial table may be secured or clamped directly to the catheter laboratory table via one or more securement mechanisms (e.g., clamps, etc.) aligned along the angled edge. While other variations include radial table assemblies which may be secured to a separate back table.

Additional features and/or accessories may be utilized in combination. For instance, a radiation shield may be attached along the radial table edge. The radiation shield may be attached along any of the edges of the table assembly in proximity to the surgeon or practitioner to provide for additional shielding against, e.g., x-rays, which may be emitted by imaging devices typically used to image the patient's body. Other features may include one or more cushion supports which are configured and shaped for use along the cradle member may be provided with the radial table assembly or separately. Additionally, surgical drapes configured for use with the table assembly or any number of instruments or displays which are positionable upon the back table or radial table may also be provided, if so desired. All or any one of these accessories may be provided in a kit along with the radial table assembly or they may be provided separately.

### BRIEF DESCRIPTION OF THE DRAWINGS

FIG. 1 shows a perspective view of one variation of a radial access table positioned in proximity to a patient platform and having a rotatable support assembly for the patient's arm.

FIGS. 2A and 2B illustrate another variation of a support assembly rotatably mounted upon the radial access table.

FIGS. 3A and 3B show perspective views of a patient's arm extending from a catheter laboratory table while supported upon the support assembly and further illustrating how the patient's arm is movable relative to the stationary radial access table in a manner corresponding to a movement of the catheter laboratory table.

FIG. 4 shows an illustrative view of how an imaging system such as a fluoroscopy device is positionable relative to the radial access table.

FIG. 5 shows a perspective view of how an interface portion of the radial access table is reconfigurable relative to the remainder of the table.

FIG. 6 shows a perspective view of another variation in which an interface portion and radial table is reconfigurable to collapse relative to the back table.

FIG. 7 shows a perspective view of another variation illustrating the back table supported by a table leg which provides sufficient clearance beneath the table.

FIG. 8 shows a perspective view of an example of how the interface portion may be secured to the radial table portion.

FIG. 9 shows a perspective view of an example of how the radial table and interface portion may be reconfigurably secured to the back table.

FIG. 10 shows a perspective view of another example illustrating how the radial table and interface portion may be reconfigurably secured to the back table.

FIG. 11A shows a perspective view of another variation of a support assembly.

FIGS. 11B and 11C show end and side views, respectively, of an elbow cradle which may be made of a radio-lucent plastic for use with the radial table assembly.

FIGS. 12A and 12B show side and end views of a support arm variation for rotatably securing the support assembly.

FIGS. 13A and 13B show perspective and assembly views of another variation of a support assembly.

FIG. 14 shows a perspective view of the support assembly of FIG. 13A positioned upon a radial access table.

FIGS. 15A and 15B show side and perspective views of yet another variation of the radial access table which may be secured to a catheter laboratory table.

FIG. 15C shows a perspective view of another variation in which a support leg may be pivotably or retractably connected to the radial table.

FIG. 15D shows a perspective view illustrating an example of how the table of FIG. 15A may be secured to the catheter laboratory table.

FIGS. 16A and 16B show perspective views of yet another variation of the radial access table which may be clamped or secured to a back table.

FIGS. 16C and 16D show perspective views of the table of FIG. 16A secured to a back table and positioned in proximity to a catheter laboratory table.

FIG. 17 shows a perspective view of a radial access table incorporating an optional drape or shield.

FIG. 18 shows an assembly view of a radial access table and optional accessories which may be specially configured for use with the table.

FIG. 19 shows a top view of a body drape which may be used with the radial table assembly.

FIG. 20 shows a top view of a table drape which may be used with the radial table assembly.

FIG. 21 shows a perspective view of the radial table assembly configured for positioning against a left side of the catheter laboratory table for accessing the vessels through the patient's left arm.

FIGS. 22A and 22B show perspective views of the catheter laboratory table being repositioned relative to an imaging apparatus for facilitating positioning the radial table assembly.

FIGS. 23A and 23B show top views of another variation of the radial table assembly having an adjustably removable interface portion and/or radial table portion.

FIGS. 23C and 23D show detail perspective views of variations for the adjusting the interface portion relative to the radial table portion.

FIGS. 24A to 24C show top views of various configurations for the adjustably removable interface portion.

FIG. 25 shows a top view of a sheath having adhesive wings which may be used with the radial table assembly.

FIGS. 26 to 28 show side, top, and front perspective view of an ergonomic hand and catheter rest which may be used with the radial table assembly.

FIG. 29 shows a top perspective view of an elbow cushion which may be used with the radial table assembly.

FIG. 30 shows a top perspective view of a hand cushion which may be used with the radial table assembly.

### DETAILED DESCRIPTION OF THE INVENTION

In accessing a radial vessel, e.g., for an interventional procedure, the limb of the patient is typically extended away from the body and secured to provide a stable insertion pathway for any number of instruments such as catheters, guidewires, intravascular balloons, stent scaffolds, etc. The limb of the patient, such as an arm, is generally positioned to extend the wrist and expose a radial artery.

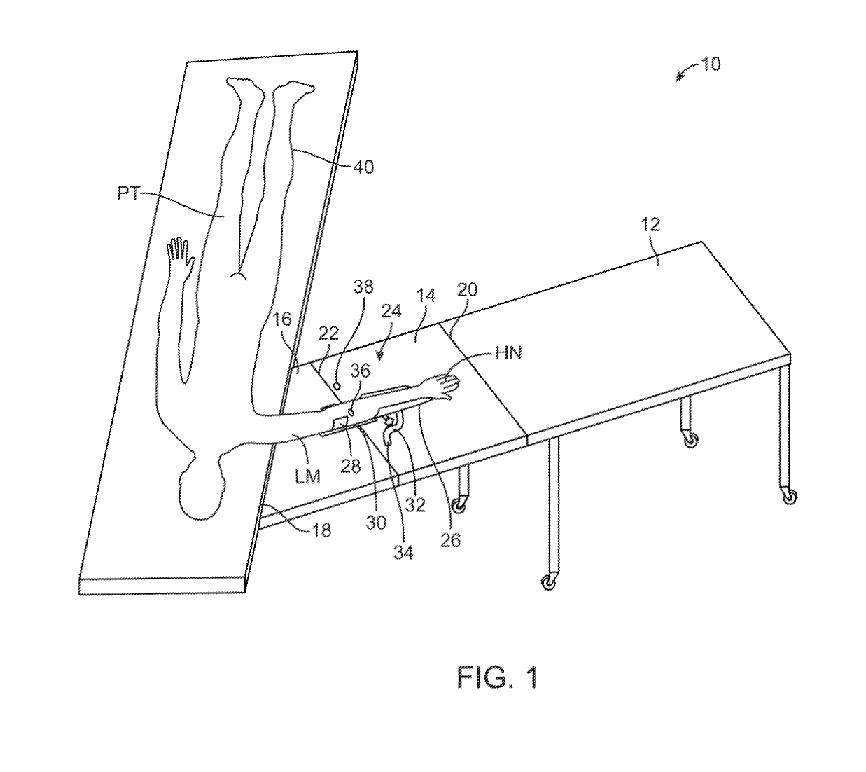

One example of a radial table assembly 10 is illustrated in the perspective view of FIG. 1 which shows the table assembly 10 generally comprising a back table 12 supported by one or more legs, a radial table portion 14 extending from the back table 12, and an interface portion 16 further extending from the radial table portion 14, e.g., in a cantilevered manner. The interface portion 16 may define an angled edge 18 which allows for the table assembly 10 to be positioned relatively at an angle and in close proximity to or adjacent to a catheter laboratory table 40 and provide a platform upon which the patient's limb LM (such as the arm) may be extended from the patient body PT positioned upon the table 40. The table assembly 10 may provide surgeons and practitioners a comfortable way to facilitate procedures, e.g., angiography, percutaneous interventions, etc., through the radial artery.

The back table 12 may comprise a surgical table or platform and the radial table portion 14 may be attached to a distal edge of the back table 12 along an optionally hinged or pivoting interface 20 and having a width of, e.g., 12-48 inches or more, and a length of, e.g., up to 20 inches or longer. The interface portion 16 may be secured at a distal end of the radial table portion 14 also along an optionally hinged or pivoting interface 22 and may have a similar width as the radial table portion 14. The interface portion 16 may extend at a distance and terminate at its distal end at the angled edge 18 which may have an angle ranging anywhere from, e.g., 10 degrees and up to 90 degrees (or more particularly 45-55 degrees), relative to the length of the table assembly 10. While each of the back table 12 and radial table portion 14 may be comprised of various materials which are radio-opaque, e.g., stainless steel, the interface portion 16 may be comprised of a radio-lucent material which is optionally anti-bacterial or able to be disinfected, e.g., various polycarbonate materials such as Lexan® (SABIC Innovative Plastics), ultra-high-molecular-weight polyethylene (UHMW), etc. Having the interface portion 16 comprised of a radio-lucent material may allow for an imaging device such as a fluoroscope (or any other suitable imaging device) to penetrate and image the proximal upper sections of the patient's limb LM, if needed.

A cradle assembly 24 may be rotatably secured to the radial table assembly 10 within any one of the connection interfaces 34, 36, 38 which may be aligned along, e.g., the proximal edge of the radial table portion 14 as shown. The cradle assembly 24 may be optionally positioned in any of the connection interfaces 34, 36, 38 depending upon the positioning of the patient body PT along the catheter laboratory table 40. The cradle assembly 24 itself may generally comprise a cradle member 26 having a length of, e.g., up to 10 inches or more, and a width of, e.g., 6 inches or more, and which is configured to define a channel to comfortably receive the patient's wrist, forearm, and hand HN. An optional proximal cradle member 28 may extend adjustably via an extension arm 30 from the cradle member 26 to support the patient's upper arm if needed or desired. The cradle member 26 and optional proximal cradle member 28 may each be fabricated from either a radio-opaque material (such as stainless steel) or from a radio-lucent material (such as polycarbonate) to further allow for imaging of the forearm and hand of the patient if also desired.

The cradle assembly 24 may define two points of rotation or articulation to enable the rotation of the cradle member 26 relative to a support arm 32 which couples the cradle assembly 24 to the connection interface 34 and to also enable the rotation of the support arm 32 relative to the connection interface 34 and radial table 14. With the patient body PT positioned upon the catheter laboratory table 40, the limb LM and particularly the hand and wrist may be extended and secured upon a cradle member 26 with the patient's fingers optionally secured to the distal end of the cradle member 26 to extend the wrist. With the limb LM secured and optionally draped, the radial artery may be readily accessed by any number of percutaneous access or surgical procedures. The surgeon and/or practitioner may be positioned between the catheter laboratory table 40 (or operating table) and the radial table assembly 10 while any number of instruments or interventional tools may be placed or extended upon the radial table portion 14 and/or back table 12.

However, if the patient PT body requires repositioning relative to the imaging device positioned above and below the patient PT and laboratory table 40, the laboratory table 40 may be translated either along its length or width while keeping the limb LM secured to the cradle member 26. Hence, as the patient body PT moves relative to the imaging device and relative to the radial table assembly 10, the patient's limb LM may also be repositioned in a manner corresponding to the repositioning of the patient body PT due to the rotation of cradle member 26 (and the hand and wrist positioned upon the cradle member 26) relative to the support arm 32 and also the rotation of the support arm 32 relative to the radial table portion 14 without interference with the surgeon or practitioner. These two points of rotation of the cradle member 26 and the support arm 32 relative to the radial table may allow for a full 360 degrees or movement. Alternatively, the rotation of one or both of the cradle member 26 and support arm 32 relative to the radial table portion 14 may be limited to rotate within specified ranges, if so desired. In either case, because the orientation of the patient's limb LM may be automatically maintained relative to the patient's body PT despite any translation of the body PT relative to the imaging device and/or radial table assembly 10, any instruments or tools extending from the patient's limb LM and laid upon the back table 12 may be left in place rather than moved to accommodate movement of the patient body PT.

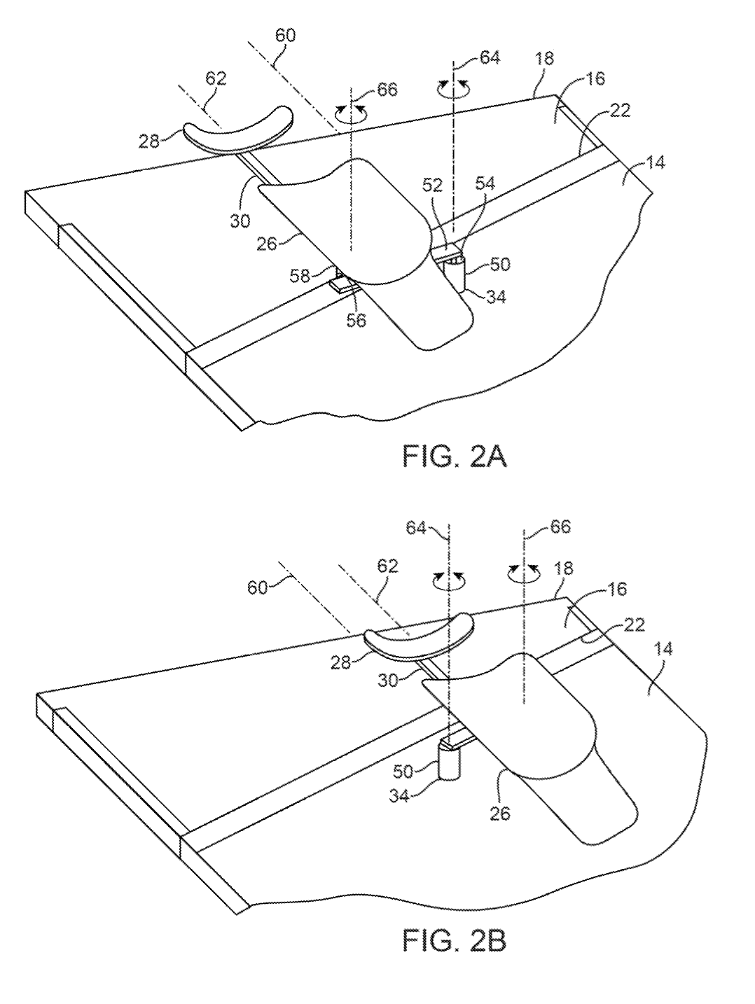

FIGS. 2A and 2B show perspective detail views (without patient limb LM shown for clarity) of another variation of the cradle assembly to illustrate its relative movement. In this variation, the cradle member 26 may be coupled to the radial table portion 14 via a support arm 52 (described in further detail below) having an insertion rod extending perpendicularly relative to the support arm 52. A receiving support 50 which defines a receiver opening 54 for receiving the insertion rod of the support arm 52 may be secured within the connection interface 34. Although a single connection interface 34 is shown in this example, any number of additional connection interfaces may be optionally placed at different locations along the radial table portion 14 to accommodate various orientations. A second receiver support 56 may extend from the cradle member 26 and define a second receiver opening 58 for receiving a second perpendicular insertion rod extending from the support arm 52.

The radial table assembly centerline 60 is shown and the cradle assembly centerline 62 is also shown for illustrative purposes. FIG. 2A shows how the cradle assembly centerline 62 is initially oriented relative to the radial table assembly centerline 60. As the laboratory table 40 and the patient body PT is repositioned relative to a stationary imaging device and stationary radial table assembly 10, the patient's arm may correspondingly cause the cradle member 26 to rotate about its cradle axis of rotation 64 as well as the cradle member axis of rotation 66. Even in its reoriented position shown in FIG. 2B, the orientation of the cradle assembly centerline 62 is still maintained relative to the radial table assembly centerline 60.

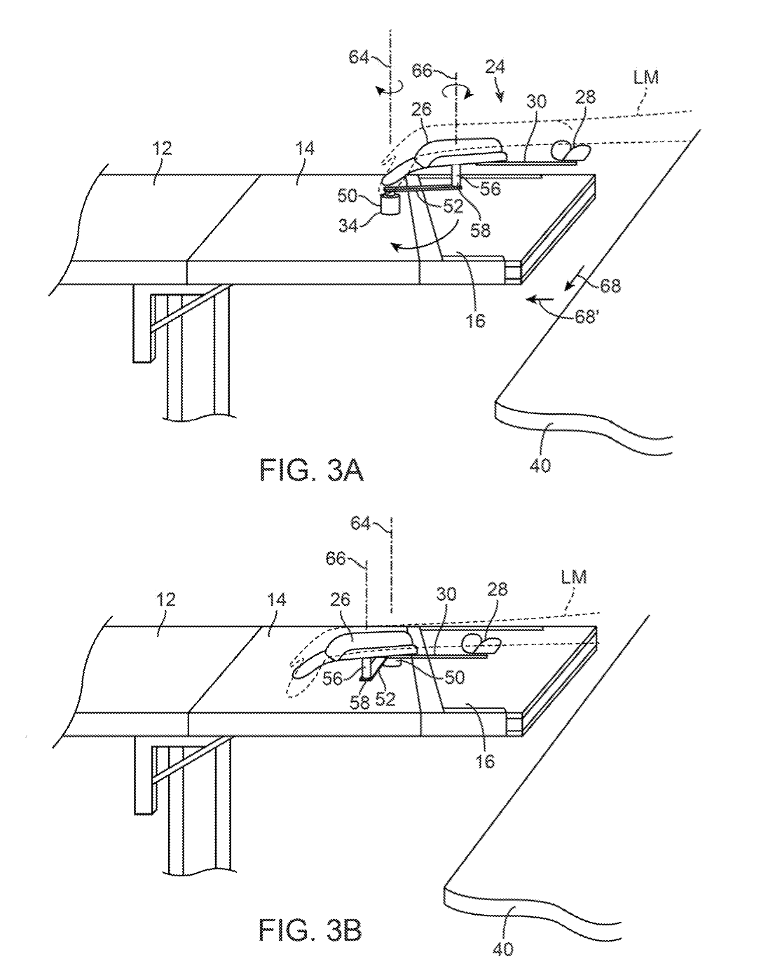

Another example is shown in the perspective views of FIGS. 3A and 3B which illustrate the radial access table assembly 10 placed against or in proximity to the laboratory table 40. An example of the patient's limb LM extending from the laboratory table 40 may be seen initially secured upon the cradle member 26. As the laboratory table 40 is translated, e.g., in a direction of motion 68 and 68′, the cradle member 26 may correspondingly rotate about its cradle axis of rotation 64 and its cradle member axis of rotation 66 such that the cradle member 26 is repositioned relative to the radial table portion 14 but an orientation of the limb LM is maintained relative to the patient body PT by the maintained orientation of the cradle member 26.

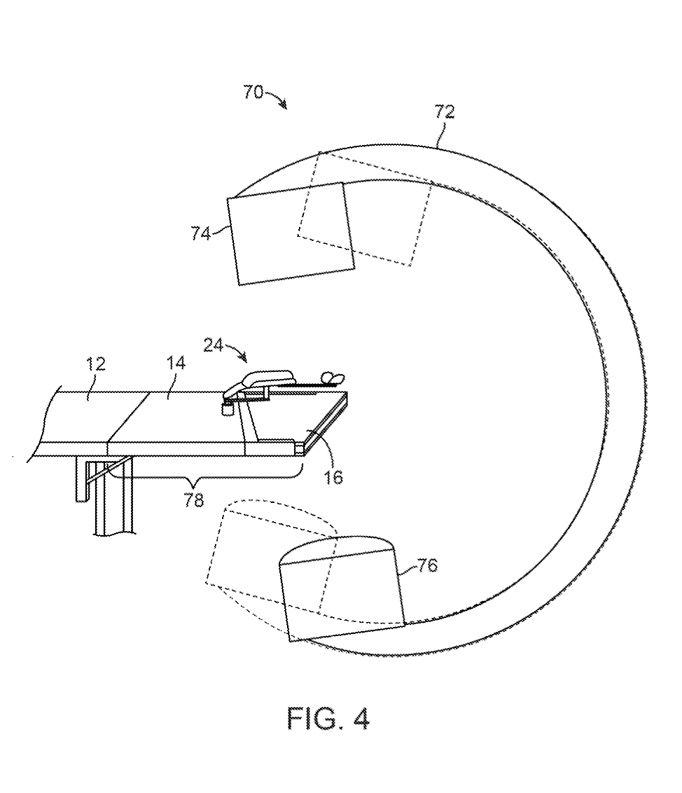

Because the radial table assembly 10 may be utilized with a number of different imaging devices, the assembly 10 may be designed to accommodate an imaging system 70 such as a fluoroscope, as shown in the perspective view of FIG. 4. Such systems 70 typically utilize a C-arm 72 having a detector 74 and x-ray transmitter 76 positioned in apposition to one another at either end of the C-arm 72. While the imaging system 70 is generally stationary relative to the laboratory table 40 and radial table assembly 10, the C-arm 72 may be rotatably adjustable for obtaining images of the underlying patient body at various angles. To accommodate such movement of the imaging system 70, the radial table assembly 10 may be configured to have the radial table portion 14 and interface portion 16 extend at a distance, e.g., anywhere from a foot or up to several feet (or more particularly between 5-6 feet), from the back table 12 in a cantilevered manner to provide for sufficient clearance distance 78 beneath the table in the event that the transmitter 76 (or any other instrument) is positioned beneath the assembly 10. This cantilevered feature may be incorporated into any of the various embodiments of the table assembly shown or described herein. Measurements and dimensions with respect to the radial table assembly are provided as illustrative examples and may be varied or customized to suit any number of patients as desired.

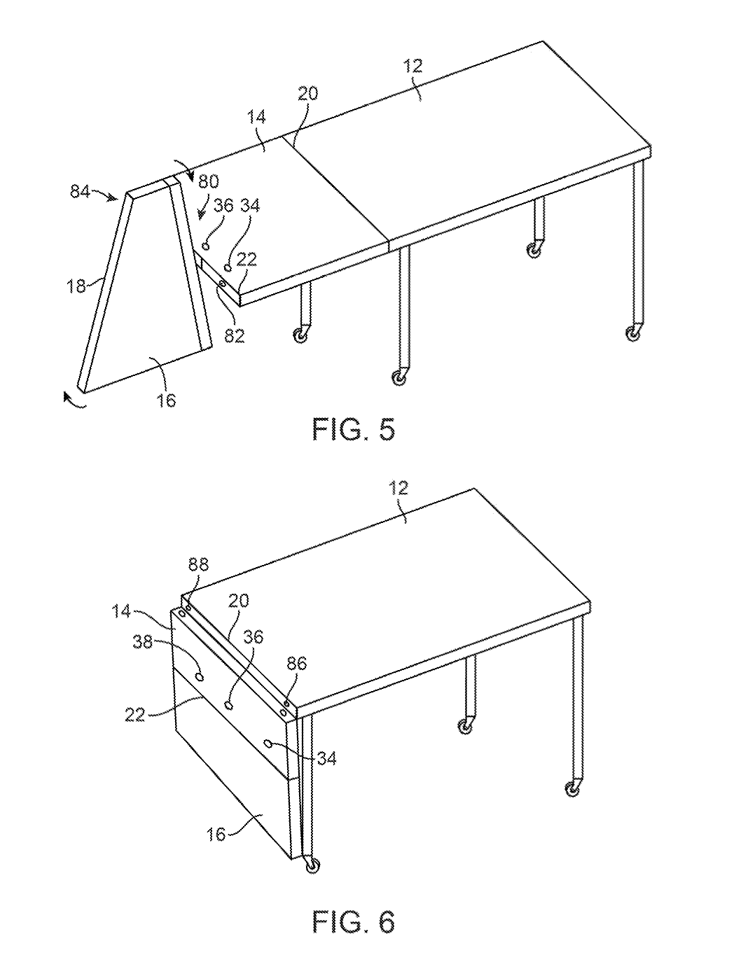

Another feature of the table assembly 10 is shown in the perspective view of FIG. 5 which illustrates a reconfigurable interface portion 16. Because the interface portion defines an angled edge 18, the interface portion 16 may be adjustably attached to the radial table portion 14. While the interface portion 16 may be entirely removable from the radial table portion 14, the variation shown illustrates an interface portion 16 which may be rotatably coupled to the radial table portion 14. The interface portion 16 may be coupled to the radial table portion via a point of rotation 80 which allows the angled edge 18 to be reconfigured into an opposite direction to enable the placement of the radial table assembly 10 on the opposite side of the laboratory table 40, e.g., from a left side to a right side of the laboratory table 40, to facilitate access through either arm of the patient. One or more mating features 82, 84 may project along the interface 22 to secure the interface 22 and prevent free rotation of the interface portion 16 relative to the radial table portion 14 during use.

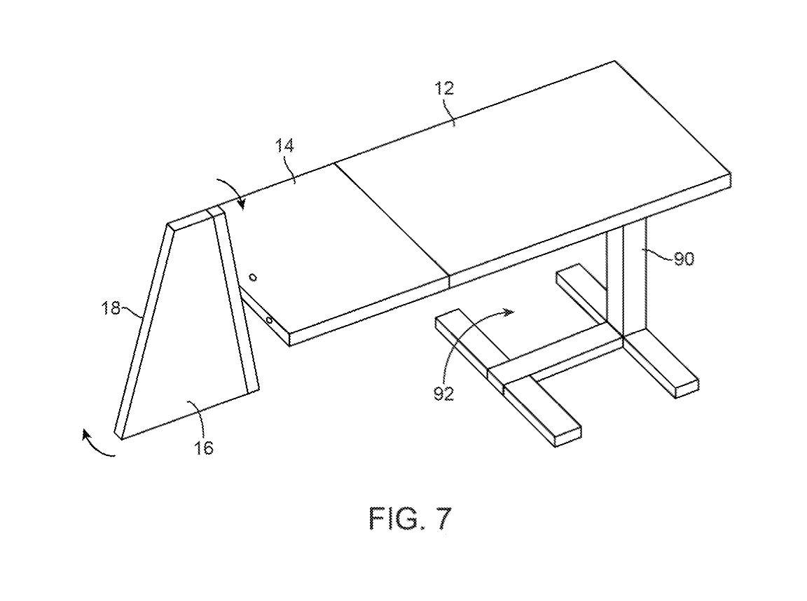

Yet another feature is shown in the perspective view of FIG. 6 which shows how the radial table portion 14 may be retractably folded along its interface 20 relative to the back table 12. By unlocking the radial table portion 14 from the back table 12, both the table portion 14 and interface portion 16 may be folded via one or more pivots or hinges along interface 20 for storage or in the event that only the back table 12 is to be utilized for a procedure. One or more radial table mating feature 86, 88 may project along the interface 20 to secure the interface 20 between the radial table portion 14 and the back table 12 when in use.

It is intended that both the rotatable interface portion 16 shown in FIG. 5 as well as the pivoting radial table portion 14 may be incorporated together or individually with any of the embodiments described herein and in any combination as practicable. For instance, each of the features shown and described in FIGS. 3A-3B as well as FIGS. 4-6 may be combined into a single embodiment of a radial table assembly, if so desired.

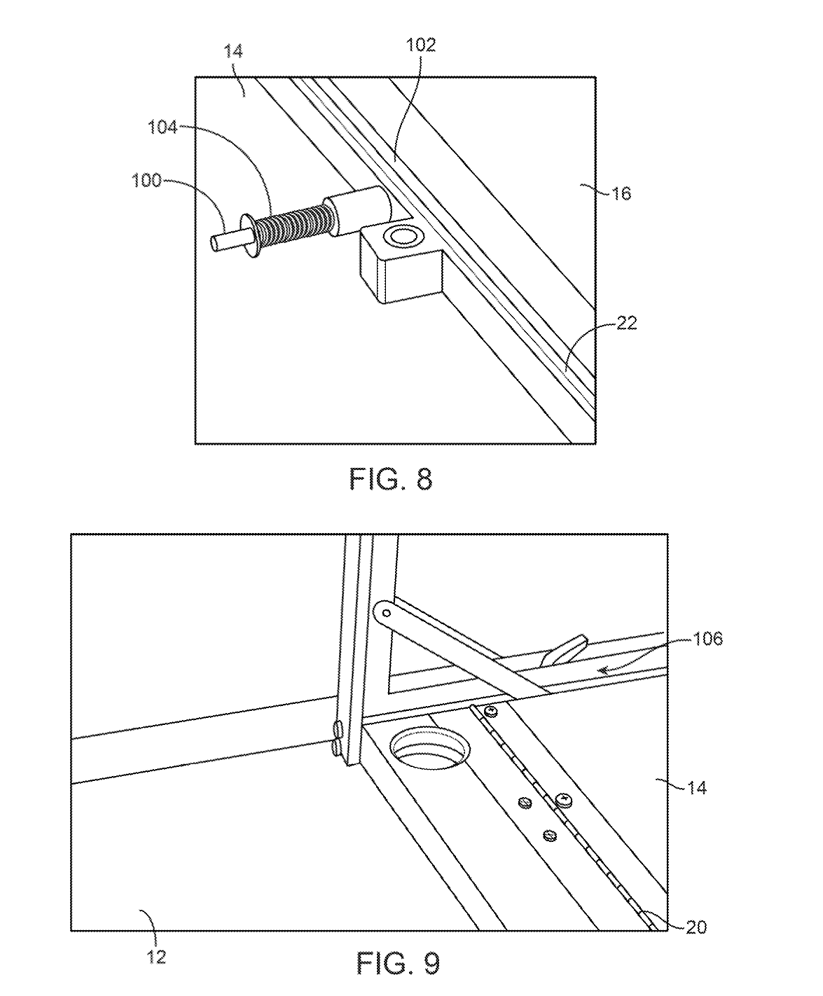

FIG. 7 shows a perspective view of yet another variation of the radial table assembly. In this example, the back table 12 may utilize a single table support 90 which projects perpendicularly from a distal end of the back table 12. Positioning of the table support 90 at the distal end may provide for sufficient clearance space 92 beneath the radial table portion 14 and interface portion 16 as well as beneath the back table 12 as well.

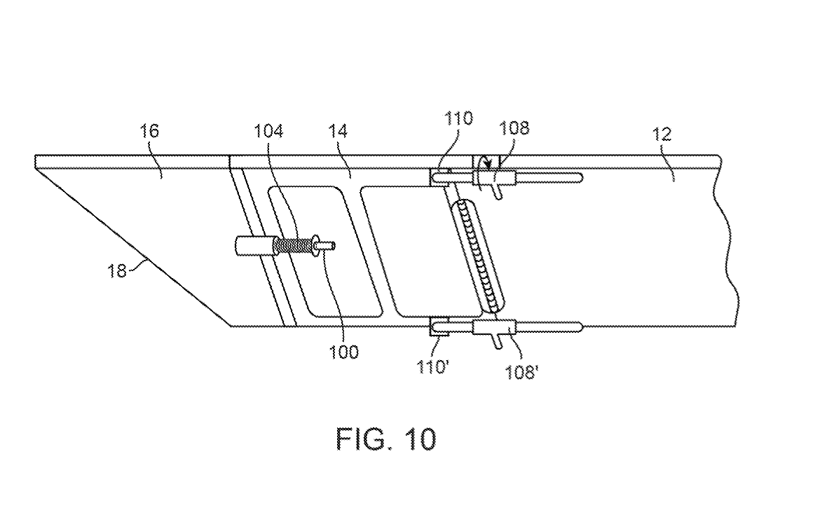

FIG. 8 shows a perspective view of one variation for implementing the pivoting point of rotation shown above in FIG. 5 between the interface portion 16 and radial table portion 14. A support member 100 may extend rigidly from the interface portion 16 for rotatable insertion within a receiving collar 102 defined along the radial table portion. A distal portion of the support member 100 may extend beyond the collar 102 to provide for a biasing member 104, e.g., a spring, to be secured therebetween. The biasing member 104 may provide for a biasing force to keep the interface portion 16 against the radial table portion 14 while still allowing for the rotation of support member 100 and interface portion 16 about collar 102.

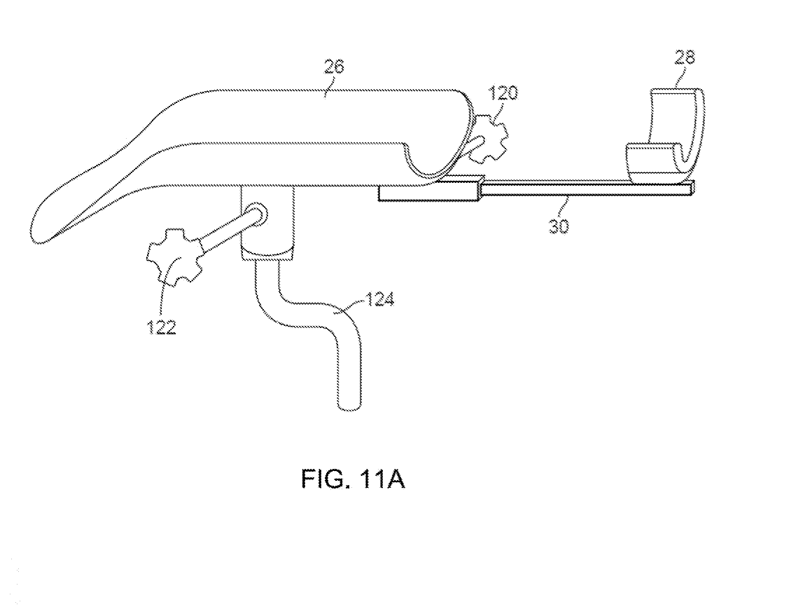

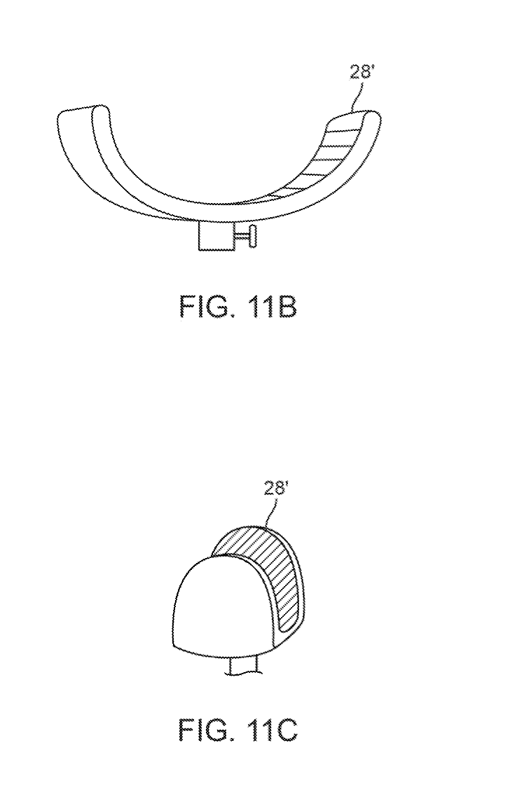

Aside from the rotation of the interface portion 16, FIG. 9 shows a perspective view of one variation for implementing the pivoting interface between the radial table portion 14 and the back table 12 shown above in FIG. 6. In this variation, locking brackets 106 may be secured between the radial table portion 14 and back table 12. Such brackets 106 may be hinged to selectively provide for repositioning of the radial table portion 14 in its cantilevered position as well as it retracted position. FIG. 10 shows another variation for implementing a pivoting interface between the radial table portion 14 and the back table 12. In this variation, one or more sliding bolts 108, 108′ may retractably extend from or to the back table 12 for securement within a corresponding receiving channel 110, 110′ defined along either the radial table portion 14 or back table 12. When the one or more sliding bolts 108, 108′ are retracted, the radial table portion 14 may be lowered but when the one or more sliding bolts 108, 108′ are extended, the radial table portion 14 may be locked into its cantilevered configuration.

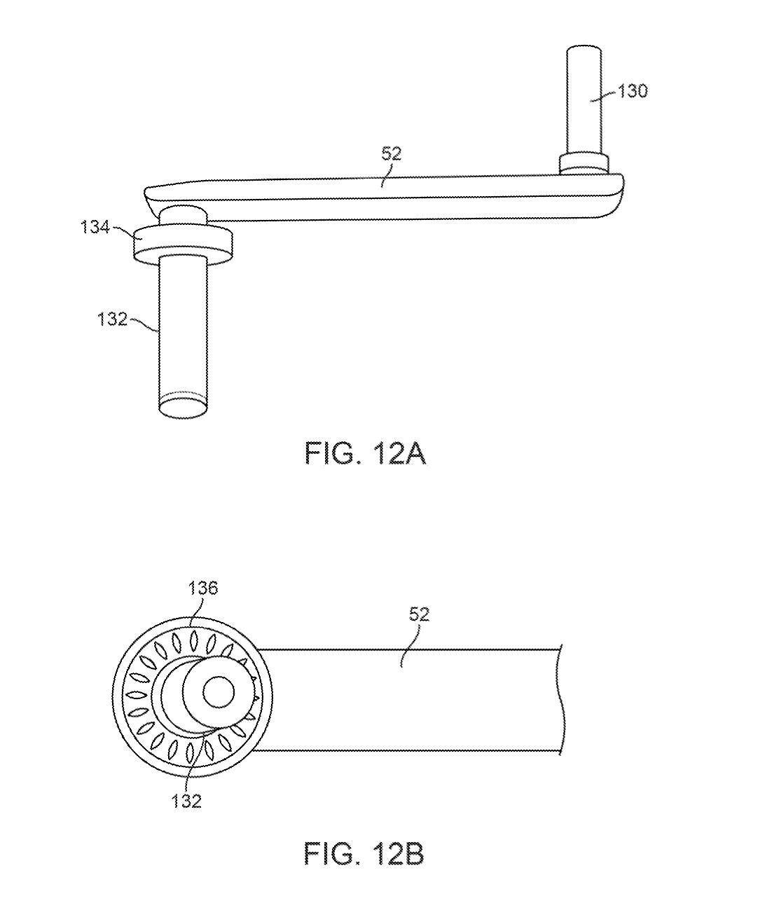

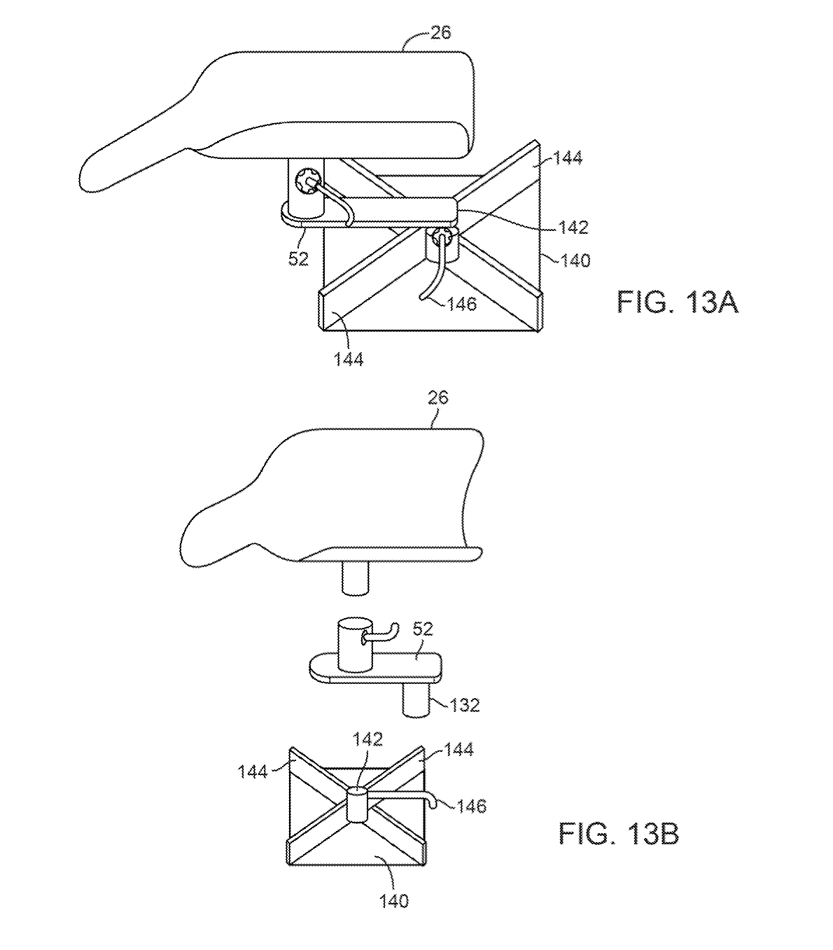

Turning now to the cradle assembly, FIG. 11A shows a perspective view of another variation of the cradle assembly having one or more optional locking controls 120, 122. Such a locking control 122 may provide for either free or tensioned rotation of the cradle member 26 or full locking of the cradle member 26 if needed. The locking control 120 may also provide for selective locking of the extension arm 30 when adjusting a position of the proximal cradle member 28. Also shown in this variation is a curved support arm 124 which may rotatably extend between cradle member 26 and the connection interface defined within the radial table portion. The support arm 124 may be an arcuate arm which can be curved and lengthened to vary the height of the cradle member 26 from the underlying radial table portion 14.

While the cradle member 26 and proximal cradle member 28 may be made from various materials such as stainless steel or other metals, one or both may be made from various radio-lucent materials such as plastic or other suitable materials. While the patient's hand, wrist, and forearm is positioned and secured along the cradle member 26, the patient's elbow or upper forearm may be positioned and secured along the proximal cradle member 28. In the event that the hand, wrist, or forearm requires imaging via the imaging system, the body portion of interest may be directly imaged without have to remove the patient's arm from the cradle and proximal cradle members 26, 28 and repositioned due to the radiolucent material of the cradle members 26, 28. FIGS. 11B and 11C show end and side, respectively, of the proximal cradle member 28′ having a span or diameter of, e.g., 6 in., and a width of, e.g., 2.5 in., fabricated from a radiolucent plastic for use with the radial table assembly. Also, because the cradle member 26 and/or proximal cradle member 28 may be fabricated from a radiolucent material such as plastic, either one or both may be removable and disposable allowing for ease of replacement.

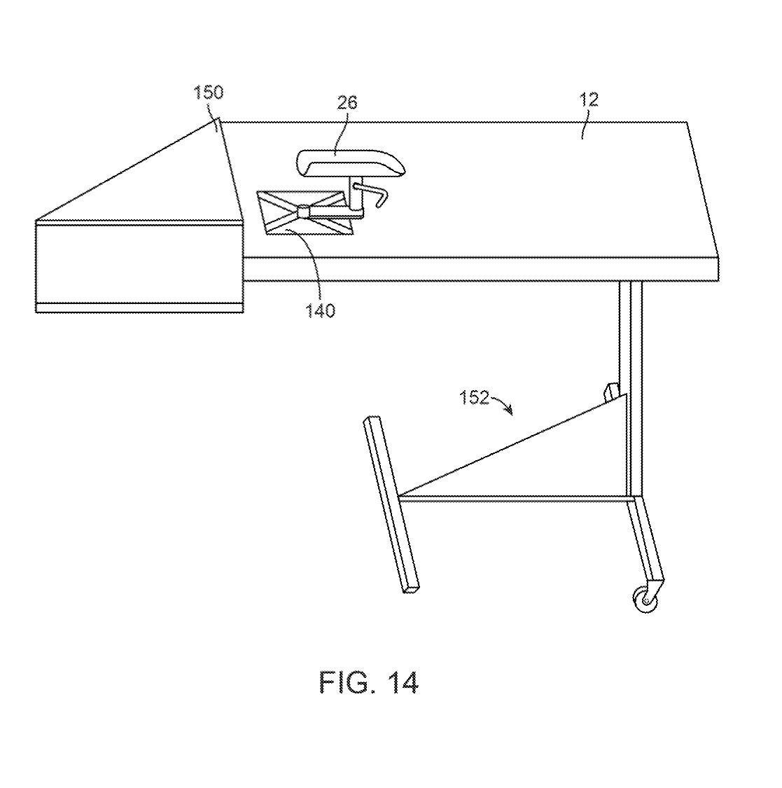

In yet another variation of the support arm, FIGS. 12A and 12B show side and end views of a support arm 52 having a rectangular cross-sectional area and a length of, e.g., about 6-7 inches. An insertion rod 130 may extend perpendicularly from the support arm 52 and may be rotatably inserted within the receiver support 56 of the cradle assembly 24. Likewise, an insertion rod 132 may extend perpendicularly from the support arm 52 in a direction opposite to insertion rod 130 and may be rotatably inserted within connection interface 34 or any one of the other connection interfaces defined within the radial table 14. An interface collar 134 may also be defined around the insertion rod 132 adjacent to the support arm for contacting against the receiving support 50. One or more contact projections 136 may also project from the interface collar 134 to facilitate rotation of the support arm 52 relative to the connection interface 34 particularly when the weight of the patient's limb LM is bearing down upon the support arm 52.

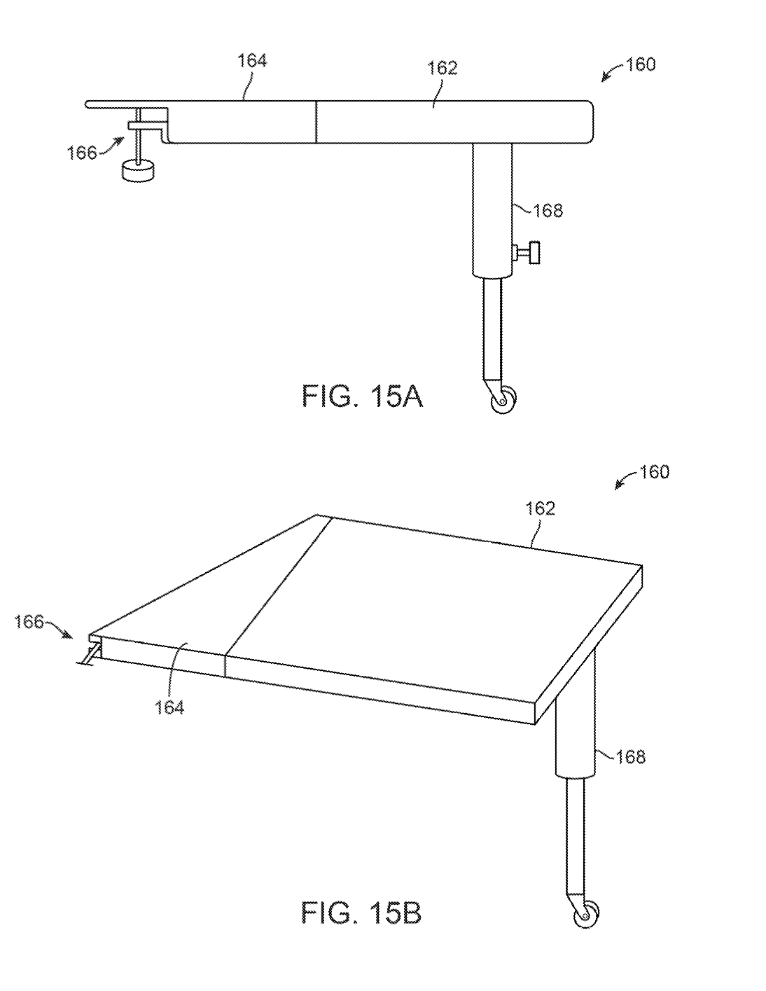

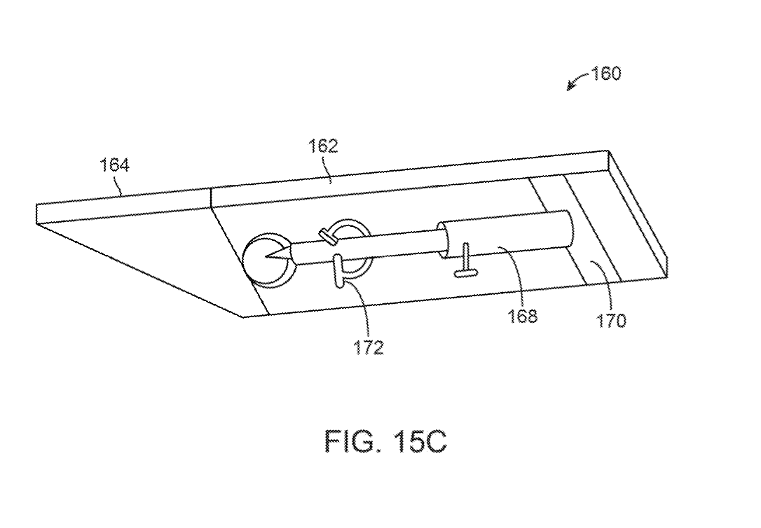

FIGS. 13A and 13B show perspective and assembly views of yet another variation of the cradle assembly. In this variation, the cradle member 26 and support arm 52 may be rotatingly coupled to a base 140 which is separate from the radial table portion 14. The base 140 shown may have one or more gussets 144 to provide for structural integrity and may further define an insertion rod channel 142 and an optional securement mechanism 146 extending from the insertion rod channel 142. With this variation, the cradle member 26 and base 140 may be positioned upon the radial table or upon another platform. FIG. 14 shows a perspective view of a radial table assembly having the cradle member 26 and base 140 positioned upon the back table 12 supported by a table support 152. The interface portion 150 is radio-lucent and sized to be thicker than the back table 12 in this variation although the thickness of the interface portion 150 may be suitably reduced.

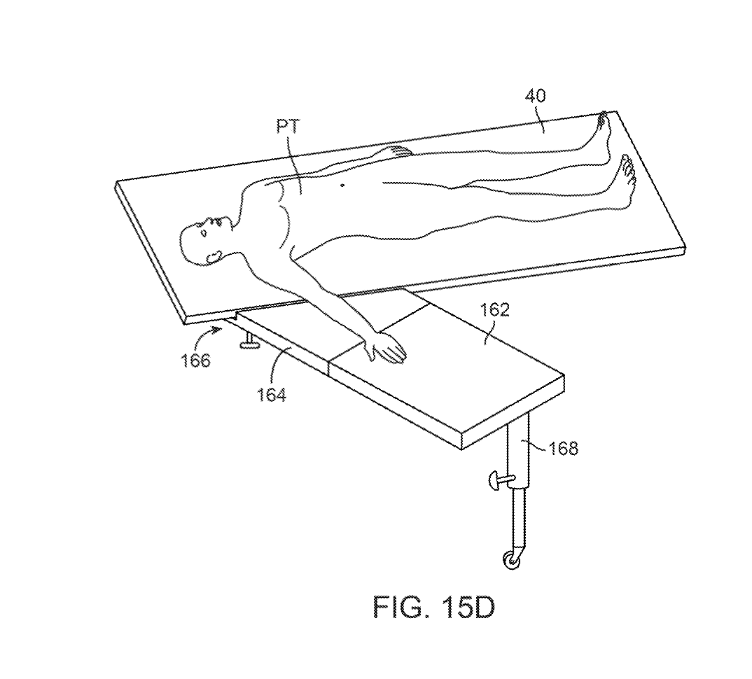

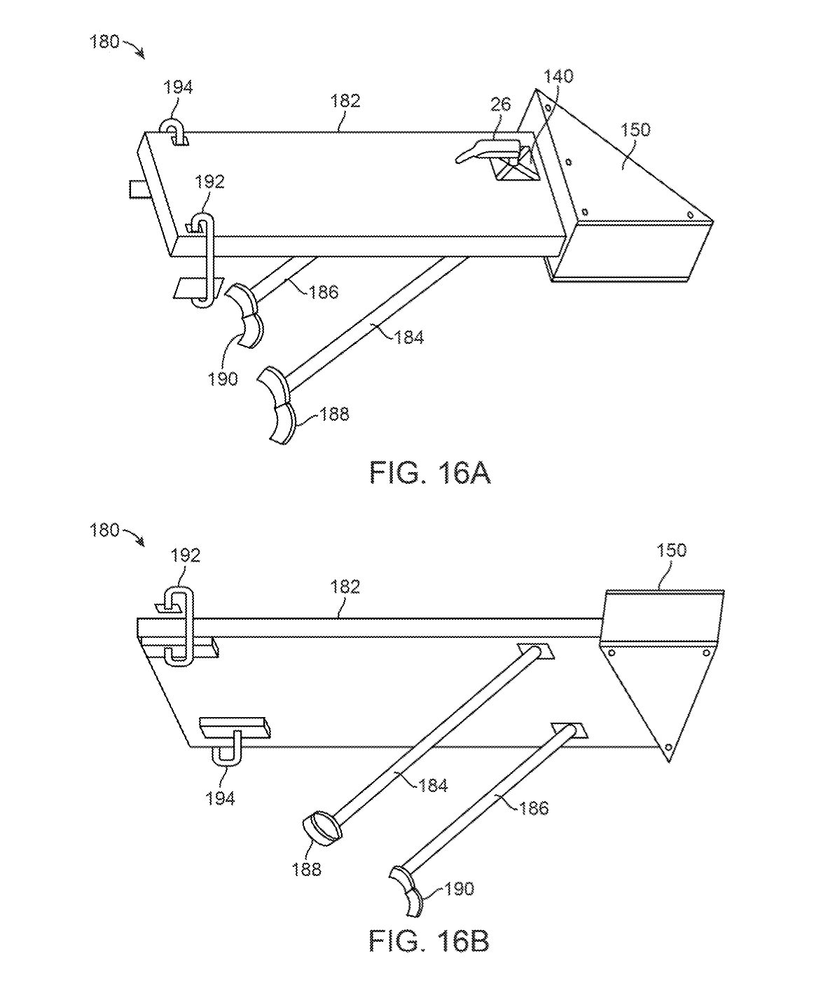

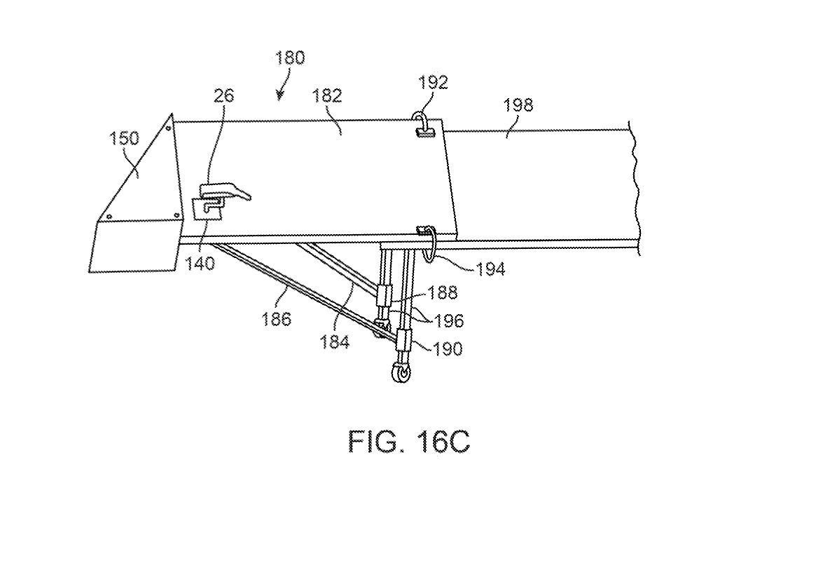

Turning now to the table assembly, FIGS. 15A and 15B show side and perspective views of yet another variation of a radial table assembly 160 generally comprised of a radial table portion 162 and interface portion 164 similar to the other variations. Yet this variation may be configured to be secured or clamped directly to the catheter laboratory table 40 via one or more securement mechanisms 166 (e.g., clamps, etc.) aligned along the angled edge. The distal end of the radial table portion 162 may be supported via a support 168 which may be coupled to the table via an optional retraction or pivoting mechanism 170 which may allow for the support 168 to be pivoted against the table portion 162 and secured by a securement mechanism 172 such as a clamp, as shown in the perspective view of FIG. 15C. FIG. 15D shows a perspective view illustrating how the interface portion 164 may be attached by the securement mechanisms 166 along a side of the laboratory table 40 to extend at an angle. The patient PT may extend their arm along the interface portion 164 to provide for radial access as needed. Also, as the laboratory table 40 moves to reposition the patient relative to an imaging device, the radial table assembly 160 may move with the table 40. With this variation, the cradle assembly may still positioned upon the radial table portion 162 for use as previously described.

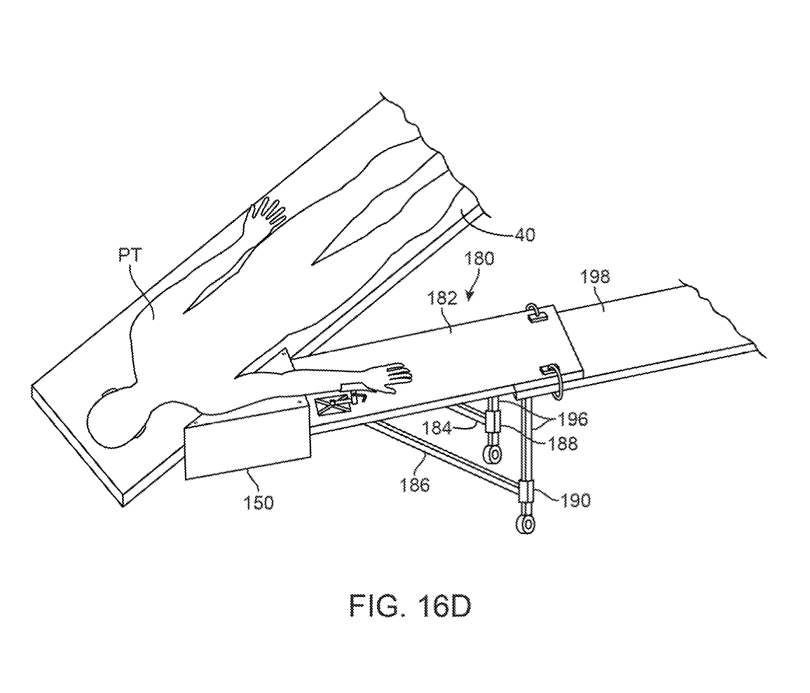

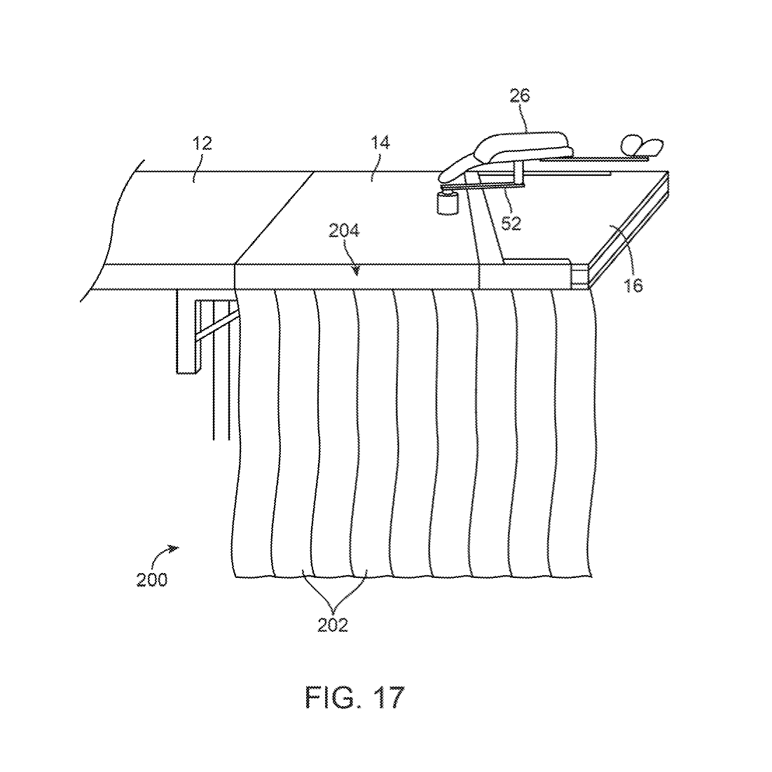

FIGS. 16A and 16B show perspective views of another table assembly variation which may be secured to a separate back table. The radial table assembly 180 shown may generally comprise a radial table 182 having the interface portion 150 and securement mechanisms 192, 194 for securement to the back table as well as support arms 184, 186 which may extend, e.g., at an angle from the radial table 182 for further securement to the legs or supports of the back table via corresponding clamping mechanisms 188, 190. FIG. 16C illustrates how the radial table 182 may be clamped to a separate back table 198 via the securement mechanisms 192, 194. The support arms 184, 186 may also be seen extending from the radial table 182 for attachment to the table legs 196 of the back table 198. In use, as shown in the perspective view of FIG. 16D, the radial table assembly 180 attached to the back table 198 may be placed against the laboratory table 40 with the angled interface portion 150 positioned against the table 40. The cradle assembly may be positioned upon the radial table 182 or other rotatable secured to the radial table 182 for use in the manner described herein with the patient PT.

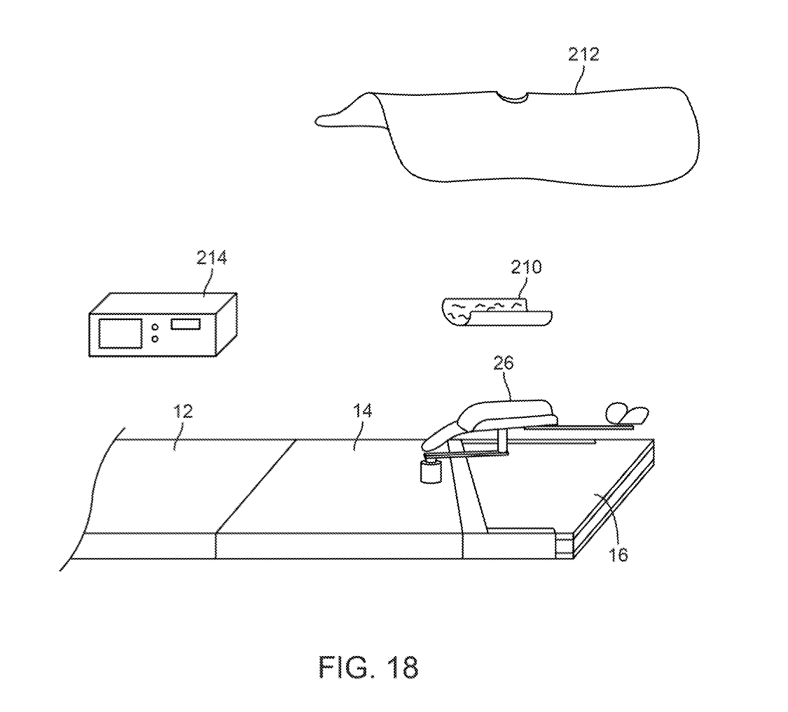

Aside from the table assembly, additional features and/or accessories may be utilized in combination. One example is shown in the perspective view of FIG. 17 which shows a radiation shield 200 which may be comprised of a single curtain or sheet or a number of individual shielded strips 202 which may be attached along the radial table edge 204. The radiation shield 200 may be attached along any of the edges of the table assembly in proximity to the surgeon or practitioner to provide for additional shielding against, e.g., x-rays, which may be emitted by imaging devices typically used to image the patient's body.

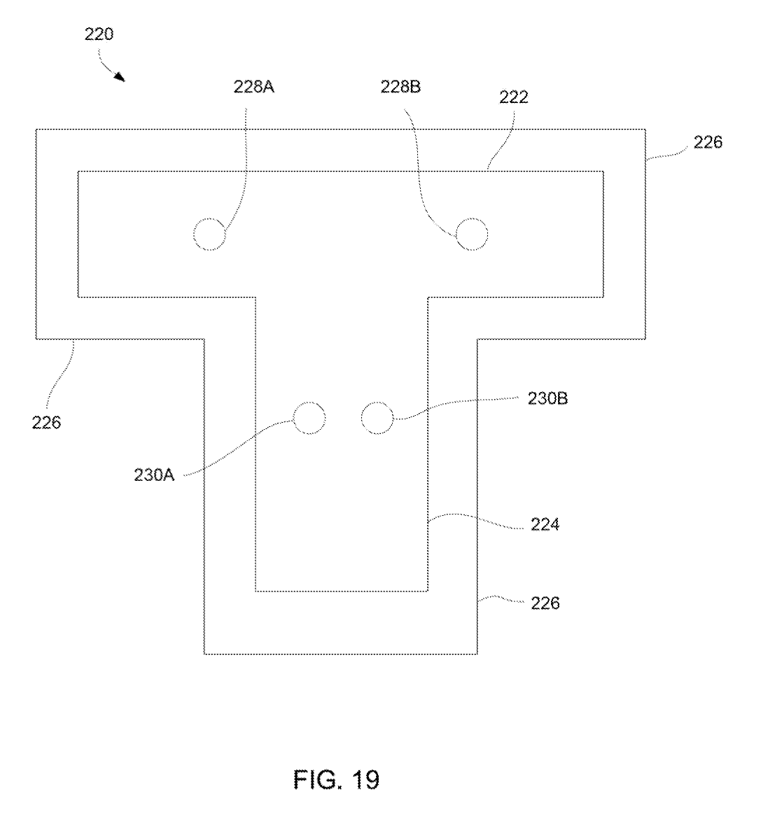

FIG. 18 shows an assembly view of additional features and/or accessories which may also be used in combination with the radial table assembly. For instance, one or more cushion supports 210 which are configured and shaped for use along the cradle member 26 may be provided with the radial table assembly or separately. Additionally, surgical drapes 212 configured for use with the table assembly or any number of instruments or displays 214 which are positionable upon the back table 12 or radial table 14 may also be provided, if so desired.

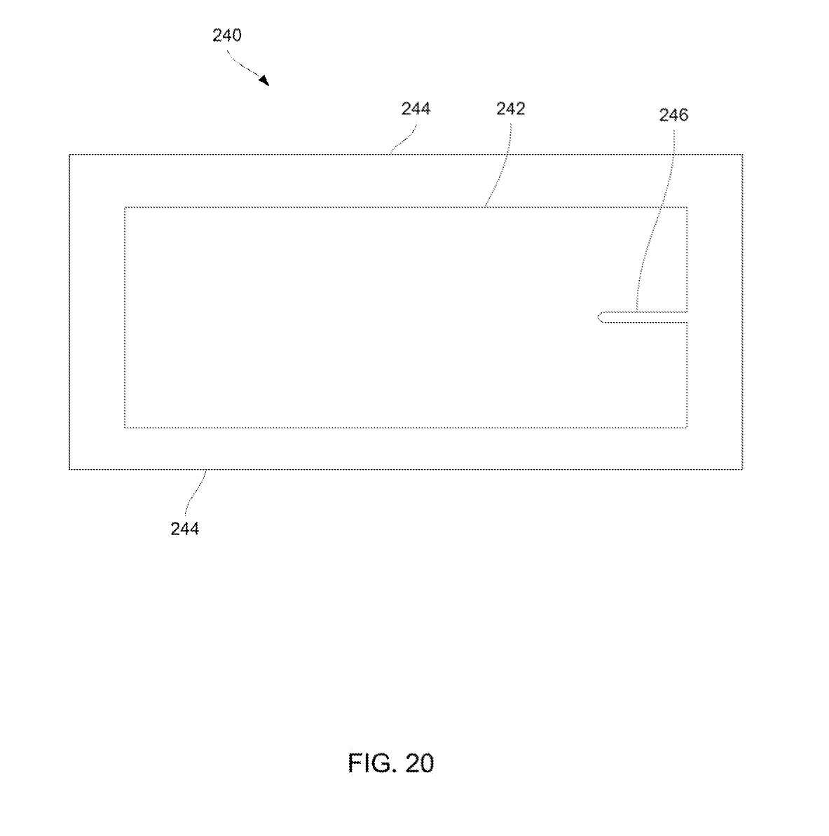

One particular variation of a surgical drape 220 which is configured for use with the radial table assembly is shown in the top view of FIG. 19 which shows body drape 220 which is splayed out. The body drape 220 may be designed for both radial and/or femoral access such that the drape 220 forms a T-shape configuration. A central portion 224 may have a width of e.g., 3 ft., with a transverse portion 222 extending a further, e.g., 4 ft., in width from either side of the central portion 224 such that the overall length of the transverse portion 222 is, e.g., 11 ft. The central portion 224 may have an overall length of, e.g., 7 ft. The transverse portion 222 and central portion 224 may be formed of an absorbent material surrounded by a plastic border 226 having a width, e.g., 1 ft., around the periphery.

The openings 228A, 228B for accessing either or both of the patient's left and/or right wrist (in the case of a radial procedure) may be defined through each of the transverse portion 222 located around, e.g., 2 ft., from the outer edges on either edge while the openings 230A, 230B for accessing either or both of the patient's left and/or right femoral arteries along the legs may be defined along the central portion 224, e.g., 28 in., from the top edge of the drape 220. The dimensions shown are exemplary and may, of course, be varied depending upon the desired size and patient anatomy or requirements.

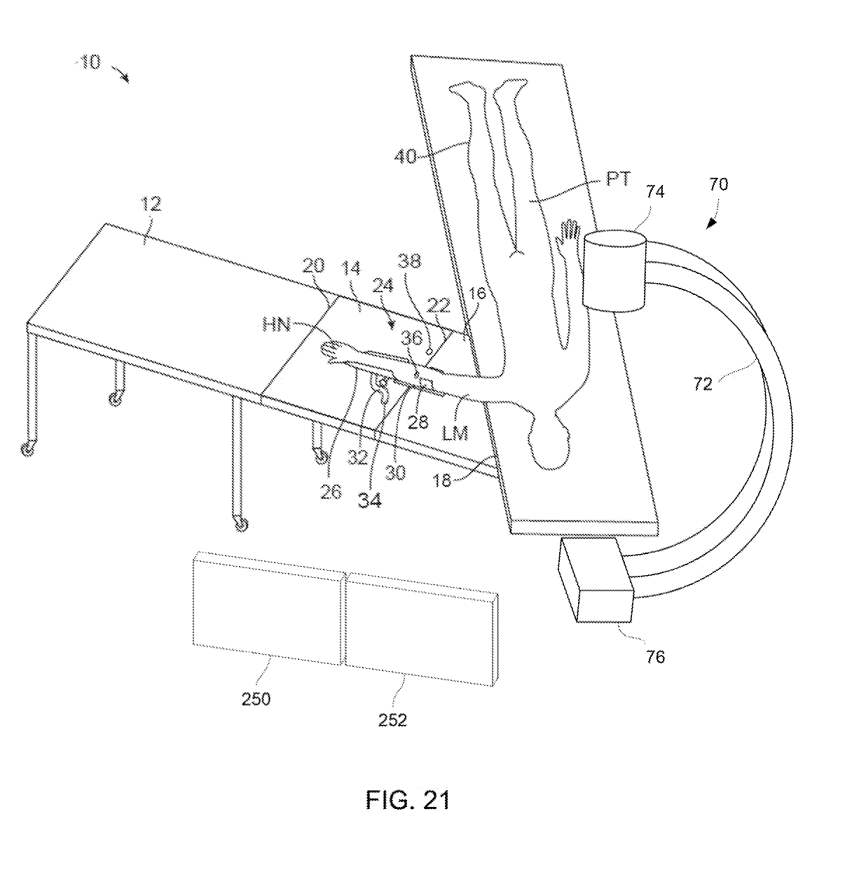

FIG. 20 shows a top view of yet another drape in table drape 240 which may be used specifically with the radial table assembly. The table drape 240 may be fabricated of an absorbent drape 242 with a border 244 of plastic, such as a clear plastic having a width of, e.g., 1 ft., around a periphery of the absorbent portion 242. The table drape 240 may have an overall length of, e.g., 9 ft. (including the border 244), and a length of the absorbent portion of, e.g., 7 ft. Furthermore, a split 246 may be defined along an edge of the drape 240 having a length of, e.g., 9 in. The dimensions shown are exemplary and may, of course, be varied depending upon the desired size and patient anatomy or requirements.

All or any one of these accessories may be provided in a kit along with the radial table assembly or they may be provided separately. Moreover, it is further intended that any of the individual features described herein may be utilized in any number of combinations with any other feature as practicable.

As previously described, the radial table assembly 10 may be configured for positioning along either side of the catheter laboratory table 40 such that the table assembly 10 is placed on either the left or right side of the patient body PT. Such flexibility facilitates a number of various trans-radial artery endovascular procedures (e.g., coronary artery angiography, peripheral artery angiography, coronary artery angioplasty and stenting, peripheral artery angioplasty and stenting, etc.).

While a trans-femoral approach is typically used as a method for percutaneous angiography and intervention, the trans-radial approach greatly facilitates the operating physician's comfort particularly in managing the numerous long wires and catheters. In particular, the radial table assembly 10 facilitates access through a patient's left radial artery. This is possible, in part, because accessing the heart through the left radial artery is similar to utilizing a trans-femoral approach in techniques and catheter instruments as many practitioners are readily familiar with such a trans-femoral approach. Furthermore, access through the patient's left radial artery may be desirable if the patient has previously had bypass surgery because access through the patient's right radial artery may be relatively more difficult because of the possibility of stents or grafts positioned within the arteries.

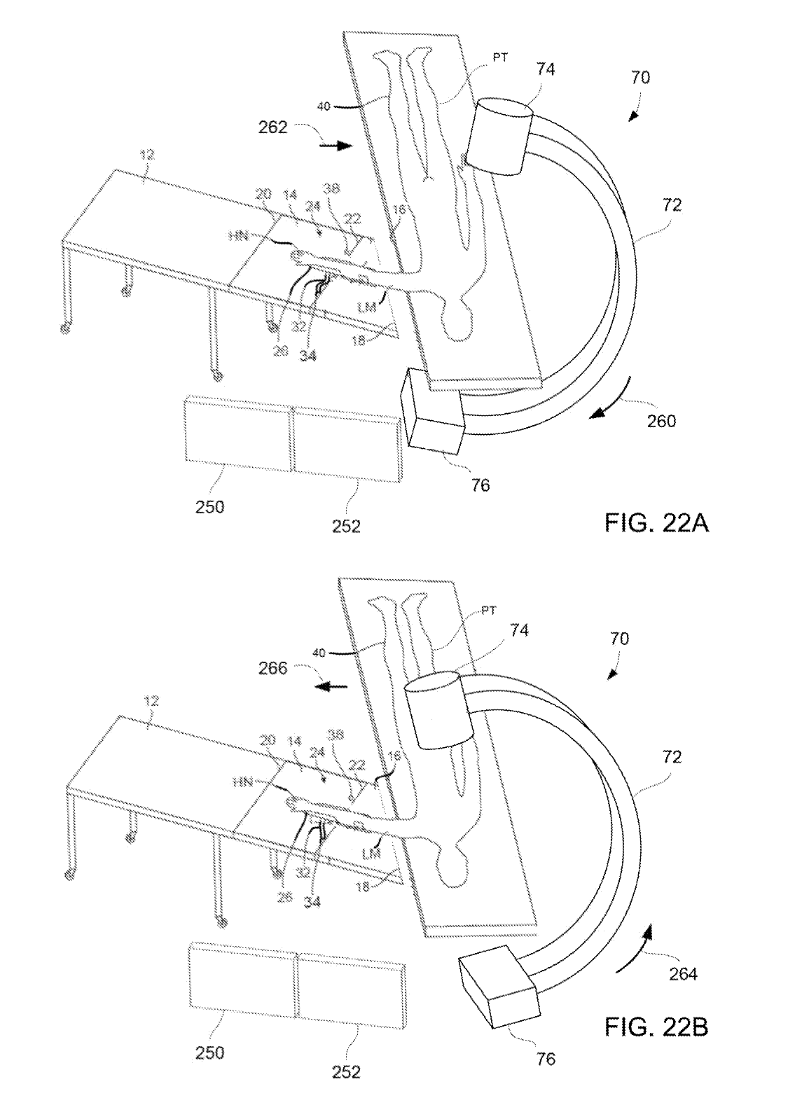

FIG. 21 shows an example of a radial table assembly 10 positioned on the left side of the laboratory table 40 and on the patient's left side. The table assembly 10 is illustrated having the interface portion 16 reversed relative to the back table 12 to allow for placement of the table assembly 10 at an angle relative to the laboratory table 40 and patient's body PT. Because the table assembly 10 is positioned accordingly, the practitioner may stand or sit between the table assembly 10 and laboratory table 40 and comfortably access the patient's left radial artery by using the practitioner's left or right hand. One or more monitors 250, 252 may be repositioned near the head of the patient PT so that the practitioner may comfortably maintain visual contact with the images on the monitors.

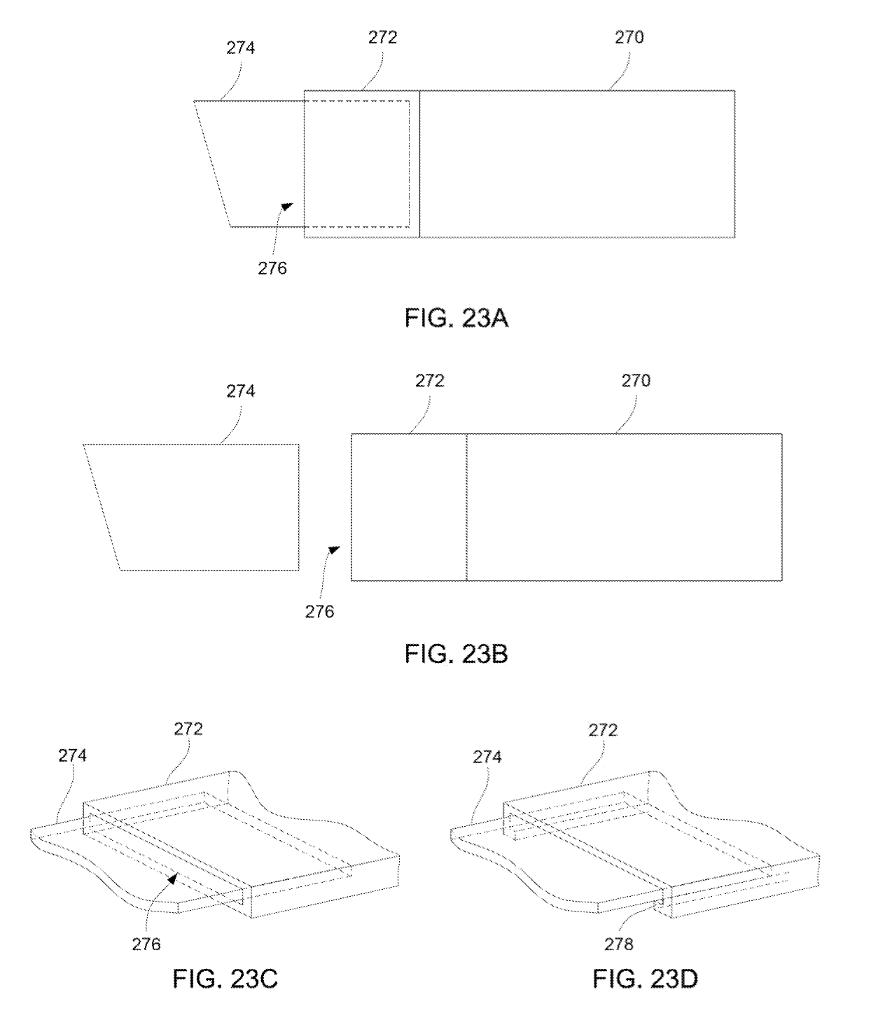

Whether the table assembly 10 is positioned on the left or right side of the patient PT, the table assembly 10 may be secured into position (e.g., locking or blocking its wheels) relative to the laboratory table 40 after the patient has been suitably positioned relative to the imaging system 70. Such a method involves iso-centering the radiographic images around the patient's heart to ensure that the projected images of the heart are readily viewable upon the monitors 250, 252 (or other viewing instrumentation) regardless of how the laboratory table 40 and/or imaging system 70 is translated or moved during a procedure. Although imaging of the patient's heart is shown and described, iso-centering for other regions of the body are readily applicable using the same or similar methods. Iso-centering images of the patient's heart may first involve positioning the table assembly 10 into proximity to the laboratory table 40. The laboratory table 40 may then be adjustably translated away from the table assembly 10 (as indicated by the direction of translation 262) with the image intensifier unit or detector 74 of the imaging system 70 rotated at an angle away (as indicated by the direction of rotation 260) from the practitioner and table assembly 10 while maintaining an image of at least the apex of the patient's heart upon one or more of the monitors, as shown in FIG. 22A. Because the patient's limb LM is positioned upon the cradle member 26 and freely repositioned, the patient PT and laboratory table 40 may be moved relative to the radial table assembly 10 without having to move the entire table assembly 10 and all the instruments positioned upon the table assembly 10. The transmitter 76 of the imaging system 70 is correspondingly rotated towards the laboratory table 40 and may become positioned beneath the table assembly 10.

The patient PT and laboratory table 40 may then be adjustably translated towards the table assembly 10 (as indicated by the direction of translation 266) with the image intensifier unit of the imaging system 70 rotated at an angle towards (as indicated by the direction of rotation 264) the practitioner and table assembly 10 such that the image intensifier unit or detector 74 is rotated towards the table assembly 10 also while maintaining the image of at least the apex of the patient's heart on the monitors 250, 252, as shown in FIG. 22B.

With the positioning of the heart centered with respect to the imaging via the imaging system 70, the position of the table assembly 10 may then be locked into place so as prevent any inadvertent movement of the table assembly 10 during a procedure. Because the table assembly 10 tracks the movement of the patient's limb LM to the patient's body via the cradle member 26, the patient PT may be freely repositioned as needed while leaving the instruments and entry point into the patient's limb LM undisturbed. Moreover, despite repositioning of the laboratory table 40 and patient PT, the image of the heart via the imaging system 70 may also remain centered.

In the variations of the table assembly described, the interface portion may be adjusted in a number of different ways. One alternative mechanism for adjusting a position of the interface portion may utilize an interface portion which is translatably adjustable relative to the remainder of the table assembly. By adjusting a position of the interface portion relative to the remainder of the table assembly, allows for a quick adjustment of the interface portion and interface edge relative to the laboratory table 40, if needed, without having move the entire table assembly. Once the interface portion has been desirably adjusted, it may be optionally locked or secured to prevent any inadvertent movement through any number of securement mechanisms.

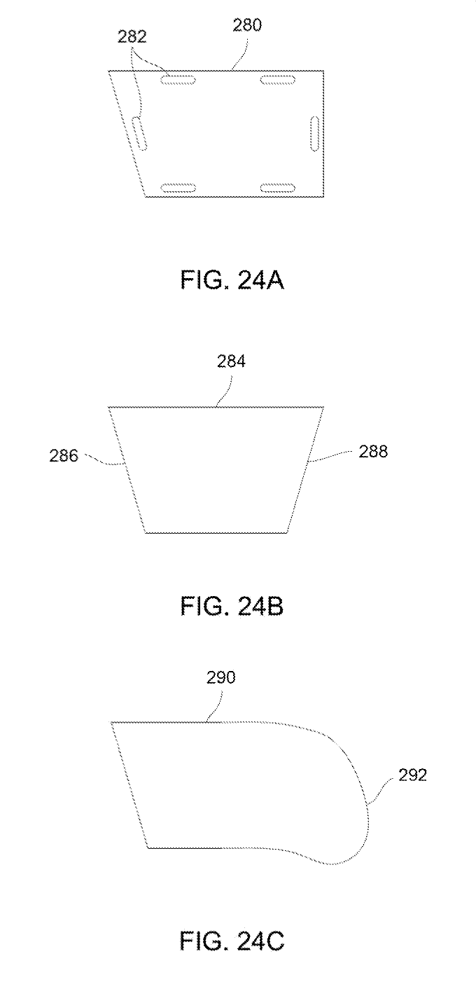

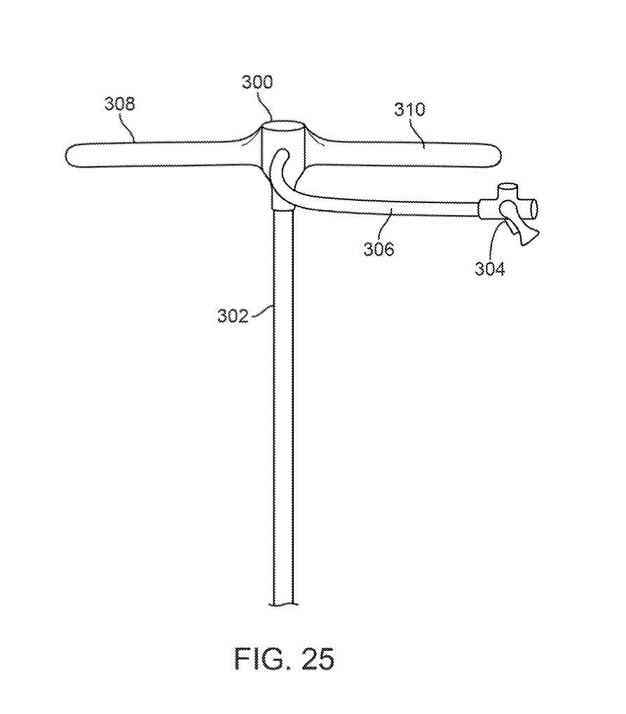

FIGS. 23A and 23B show top views of one such variation of the table assembly having an adjustably positionable interface portion 274 which is also optionally removable. The radial table portion 272 and/or back table 270 may define an opening or channel 276 which is sized to slidably receive the interface portion 274. As shown, the interface portion 274 may be adjustably moved within the opening or channel 276 without having to alter a position of the radial table portion 272 or back table 270. Optionally, the interface portion 274 may be removed entirely from opening or channel 276, as shown, and flipped to accommodate positioning of the table assembly on the opposite side of the laboratory table 40, if so desired, or the interface portion 274 may be removed from the radial table portion 272 and hung on the wall or otherwise stored for disinfection or for later use.

FIGS. 23C and 23D show detail perspective views variations for adjustably moving the interface portion 274 relative to the radial table portion. The opening or channel 276 may be optionally configured as an enveloping channel, as shown in FIG. 23C, or it may define ledges or lips 278 which extend partially beneath the interface portion 274 to provide support. Other alternative adjustment mechanisms may be incorporated into the interface as so desired.

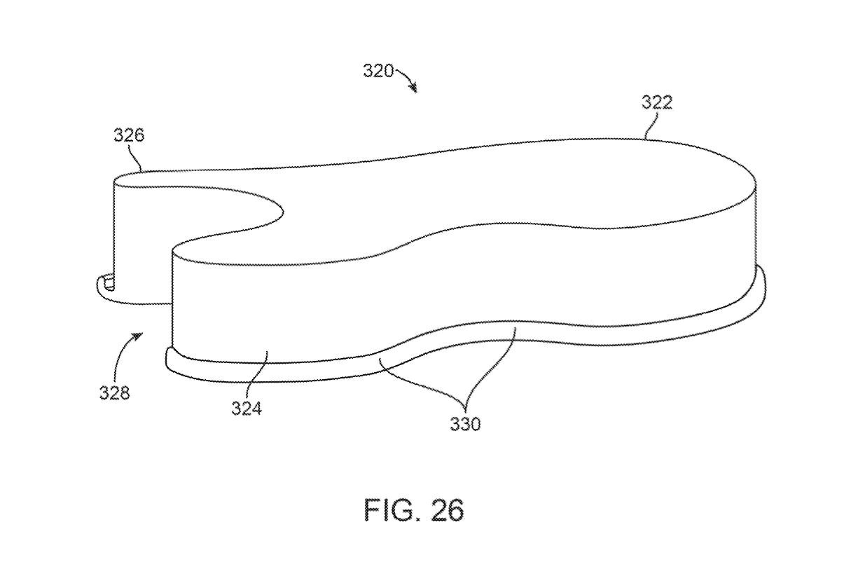

Aside from the adjustability of the interface portion, the interface portion itself may be configured into various embodiments for use with the radial table assembly. These variations may be configured to be adjustable relative to the remainder of the table or they may remain secured or otherwise integrated with the table assembly. FIGS. 24A to 24C show alternative variations of the interface portion. For instance, the variation of FIG. 24A shows a top view of an interface portion 280 which may include one or more openings 252 defined along the periphery of the interface portion 250 for serving as handles or for hanging the interface portion. FIG. 24B shows another variation of an interface portion 284 which may define angled edges 286, 288 along both ends of the interface portion 284. The angled edges 286, 288 may be angled to be parallel to one another (or similarly angled) or angled to be opposite in direction (as shown) allowing for additional flexibility in repositioning the interface portion 284 relative to the radial table portion. FIG. 24C shows a top view of yet another variation in interface portion 290 which may have one or both ends configured to have a curved or arcuate portion 292 which may be configured to enhance the ergonomics use for the practitioner and/or patient.

In addition to the various features of the table assembly, various disposable accessories may also be utilized. For instance, such disposables may include specially designed padding for the arm and wrist cradle, sterile drapes, hemostatic bands, micro-puncture kits, sheaths, etc. These accessories may be utilized with any of the variations of the table assembly described herein and they may also be utilized in any number of combinations with one another as well as with the table assembly as desired.

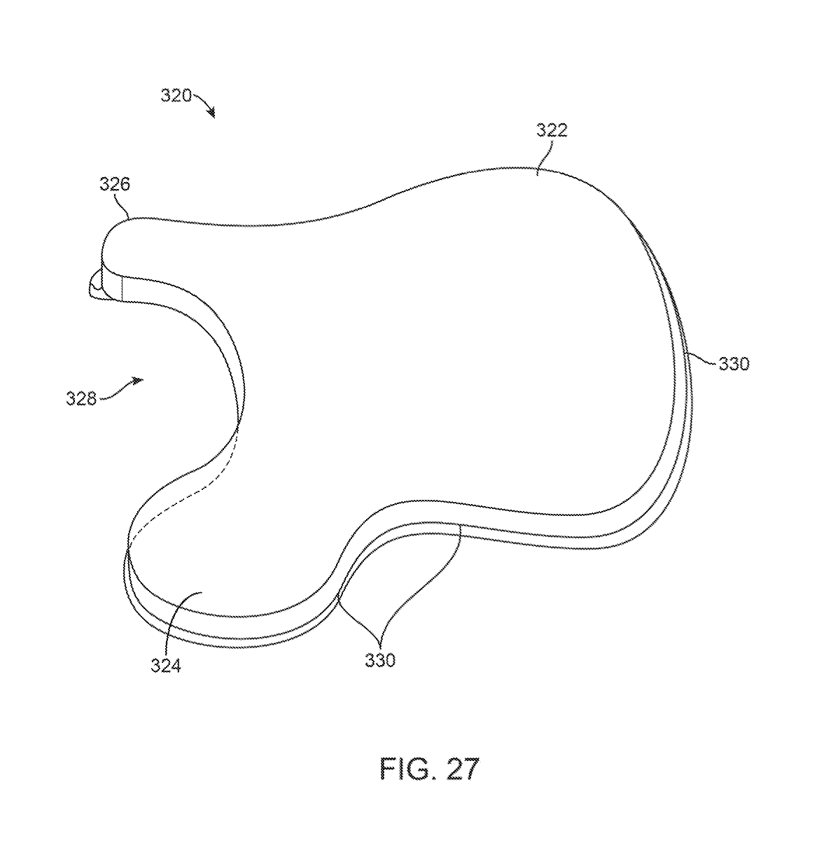

FIG. 25 shows a top view of one an example of a sheath 302 having adhesive portions 308, 310, e.g., wings or lengths which extend in a direction transverse to the sheath 302, which may be utilized. These adhesive portions 308, 310 may generally comprise adhesive portions each having a length of, e.g., 2 in., and a width of, e.g., 0.5 in, which may be incorporated with the sheath 302 to enable the sheath 302 to be adhered to the patient's skin to prevent sheath movement when any catheters are exchanged. The sheath 302 may also incorporate a junction 300 to which a port 304, such as a stopcock, may also be coupled via a fluid line 306 for optionally introducing fluids or other substances through the sheath 302 and into the patient.

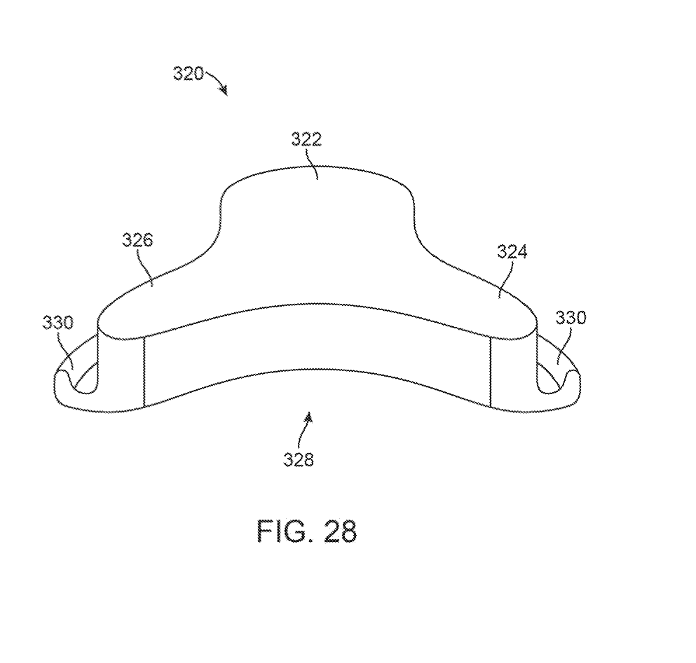

Aside from the sheath, other accessories such as supports, cushions, or rests may also be utilized in combination with the table assembly. FIGS. 26 to 28 show side, top, and front views of one example of an ergonomic hand and catheter rest 320. This rest may be positioned upon the table assembly in proximity to, e.g., cradle member 26, to provide a rest for the practitioner to place their hand and/or for an instrument or catheter to rest upon while the practitioner is performing a procedure using the table assembly. Generally, the ergonomic hand and catheter rest 320 may have an overall length of, e.g., 14 in., and a height of, e.g., 7 in., as shown in the perspective side view of FIG. 26. The rest 320 may be optionally symmetric with a first curved portion 322 extending to form two apposed second curved portions 324, 326 which extend to define concave portion 328 between the second curved portions 324, 326. The concave portion 328 may be positioned in proximity to, e.g., the cradle member 26, while the second curved portions 324, 326 extend around the cradle member 26 to accommodate the cradle member 26 and patient's hand.

The rest 320 may also incorporate a trough 330 having a height of, e.g., 0.5 in., and width of, e.g., 1 in., which extends at least partially around the periphery of the rest 320 for collecting any body fluids such as blood from the patient which may drip or flow upon the rest 320. The trough 330 may be formed around the periphery of the rest 320 and optionally along the concave portion 328. Moreover, the rest 320 may also incorporate an absorbent padding of, e.g., 0.75 in. over or upon the rest 320 to provide for added comfort to the practitioner.

FIGS. 27 and 28 show angled top and end views, respectively, of the rest 320 illustrating how the first curved portion 322 may have a length of, e.g., 10 in., extending from its distal end to the concave portion 328 and may be formed in a curved manner which is radiused to have a convex to concave contour which extend and form the two apposed second curved portions 324, 326. Each of the second curved portions 324, 326 may form an overall width of, e.g., 16 in, extending across the rest 320 with the second curved portions 324, 326 each having an individual width of, e.g., 4 in, and a length of, e.g., 5 in. The second curved portions 324, 326 may be uniform in size relative to one another or one may be formed into a larger and/or different shape relative to the other. The concave portion 328 formed between the second curved portions 324, 326 may be formed to have a diameter of, e.g., 10 in. These dimensions shown and described are exemplary and may, of course, be varied depending upon the desired size and patient anatomy or requirements.

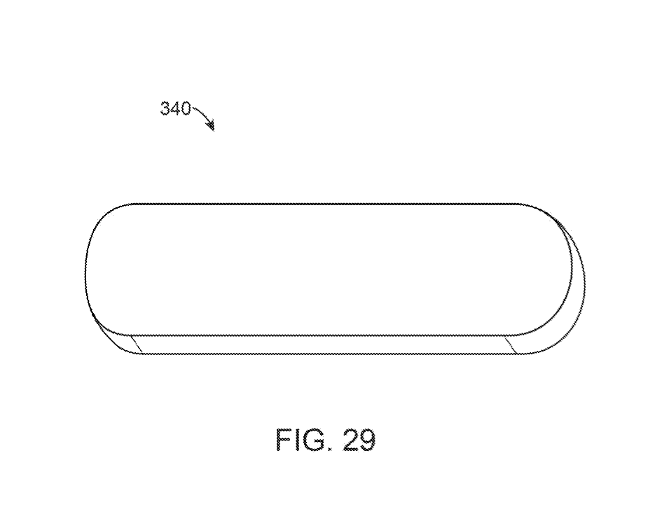

FIG. 29 shows a top perspective view of another cushion which may be used with the radial table assembly. The elbow cushion 340 shown may have a size which is specifically configured for placement upon the cradle member 26 and/or proximal cradle member 28 to allow for the patient to comfortably rest their wrist, forearm, and/or elbow. The elbow cushion 340 may be elongate with rounded edges to provide further comfort and may have a length of, e.g., 6 in., and a width of, e.g., 2.5 in., and a thickness of, e.g., 0.75 in. The dimensions shown are exemplary and may, of course, be varied depending upon the desired size and patient anatomy or requirements.

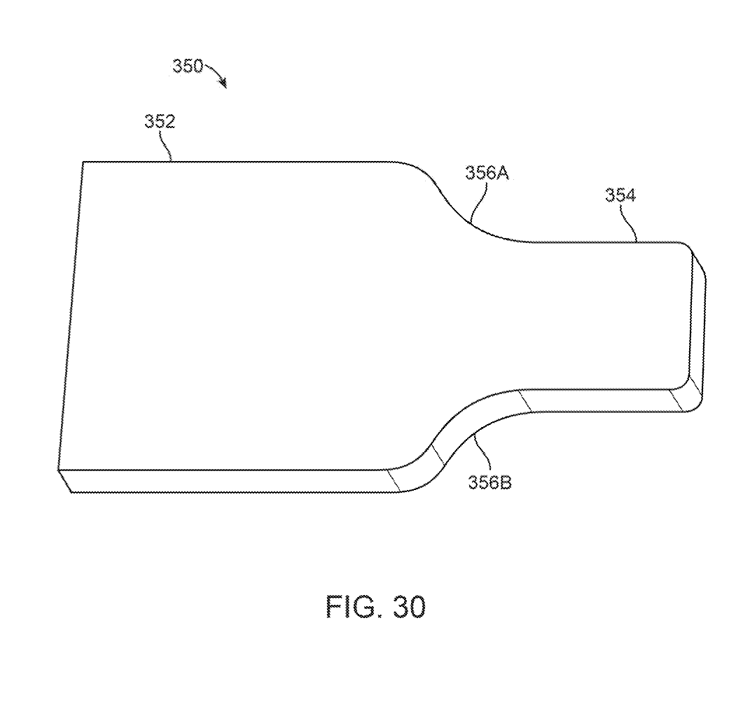

FIG. 30 shows a top perspective view of yet another cushion which is specifically configured and contoured for positioning upon the cradle member 26 to allow for the patient to rest their hand and/or wrist upon. The hand cushion 350 may have a thickness of, e.g., 0.75 in., and an overall length of, e.g., 10 in., with a first portion 352 having a first width which tapers centrally relative to a longitudinal axis along curved edges 356A, 356B to a second portion 354 having a second width which is smaller than the first width. The first portion 352 may have a first width of, e.g., 6 in., and a first length of, e.g., 6.5 in., which tapers to the second smaller portion 354 having a second width of, e.g., 3.5 in., and a second length of, e.g., 3.5 in. The dimensions shown are exemplary and may, of course, be varied depending upon the desired size and patient anatomy or requirements. The hand cushion 350 may be optionally used with the elbow cushion 340 as well as the hand and catheter rest 320 altogether or in any number of combinations. Moreover, these cushions may also be used with any of the accessories such as the drapes or other accessories described above in any number of combinations with the radial table assembly.

As previously discussed, it is intended that any of the individual features described herein may be utilized in any number of combinations with any other feature as practicable. Also, any of the apparatus and methods as described in further detail in U.S. patent application Ser. No. 14/166,556 filed Jan. 28, 2014, which is incorporated herein by reference in its entirety and for any purpose herein, may be used in any number of combinations with any of the features and methods described herein.

The applications of the devices and methods discussed above are not limited to the support of arms but may include any number of further applications. Moreover, such devices and methods may be applied to other portions of the body. Modification of the above-described assemblies and methods for carrying out the invention, combinations between different variations as practicable, and variations of aspects of the invention that are obvious to those of skill in the art are intended to be within the scope of the claims.

What is claimed is:

1. A table assembly, comprising:
a platform defining a surface which is sized to support a limb of a patient;
an interface portion extending from a distal end of the platform, wherein a position of the interface portion relative to the platform is longitudinally adjustable; and
a cradle member adjustably connected to the platform at a location proximal to the interface portion.

2. The assembly of claim 1 wherein the cradle member is configured to receive and support a limb of a patient.

3. The assembly of claim 2 further comprising:
a connection interface configured to secure the cradle member to the platform; and
a support arm rotatably extending from the connection interface to the cradle member.

4. The assembly of claim 1 wherein the interface portion is radio-lucent.

5. The assembly of claim 1 wherein the interface portion defines a distal edge which is angled relative to the platform such that the angled edge is positionable against an additional platform.

6. The assembly of claim 5 wherein the interface portion further defines a proximal edge which is angled relative to the platform.

7. The assembly of claim 5 wherein the interface portion further defines a proximal edge which is contoured.

8. The assembly of claim 1 wherein the interface portion defines one or more openings.

9. The assembly of claim 1 wherein at least a portion of the cradle is radiolucent.

10. The assembly of claim 1 further comprising a body drape which is configured for placement over the patient when placed upon an additional platform and the cradle member.

11. The assembly of claim 1 further comprising a table drape which is configured for placement upon the platform.

12. The assembly of claim 1 further comprising a hand and catheter rest which is configured for placement upon the platform in proximity to the cradle member.

13. The assembly of claim 1 further comprising an elbow pad which is configured for placement upon a portion of the cradle member.

14. The assembly of claim 1 further comprising a hand and wrist pad which is configured for placement upon the cradle member.

15. The assembly of claim 1 further comprising a sheath having adhesive wings for attachment to the limb of the patient.

16. A method of accessing a region of interest in a patient body, comprising:
positioning a left wrist of the patient body upon a cradle member which is movably attached to a first platform;
moving a second platform upon which the patient body is placed, where the first platform is in proximity to a left side of the patient body and the second platform;
moving the cradle member relative to the first platform in a manner corresponding to a movement of the second platform such that a position or orientation of the cradle member is dependent upon a position or orientation of the second platform when the left wrist is positioned upon the cradle member; and
intravascularly accessing the region of interest within the patient body via the left wrist.

17. The method of claim 16 further comprising imaging the region of interest via an imager while moving the second platform.

18. The method of claim 16 further comprising positioning a radio-lucent interface portion beneath a portion of the limb prior to moving the cradle member, where the interface portion is attached at a distal end of the first platform.

19. The method of claim 16 wherein moving the cradle member further comprises maintaining a stationary position of the first platform.

20. The method of claim 16 wherein moving a second platform comprises rotating the cradle member relative to the first platform via a connection interface rotatably attached at a distal end of the first platform.

21. The method of claim 20 further comprising rotating the cradle member relative to the first platform via a support arm rotatably extending from the connection interface to the cradle member.

22. A method of imaging a region of interest in a patient body, comprising:
positioning a table assembly having a cradle member movably coupled to the table assembly into proximity of a patient platform upon which the patient body is positionable;
moving the patient platform to a first position relative to the table assembly while imaging the region of interest at a first angle relative to the patient platform;
moving the patient platform to a second position relative to the table assembly while imaging the region of interest at a second angle relative to the patient platform, where the second position is at a distance from the first position; and,
securing a location of the table assembly relative to the patient platform.

23. The method of claim 22 wherein moving the patient platform further comprises positioning a limb of the patient body upon the cradle member which is movably attached to the table assembly.

24. The method of claim 23 further comprising moving the cradle member relative to the table assembly in a manner corresponding to a movement of the patient platform such that a position or orientation of the cradle member is dependent upon a position or orientation of the patient platform.

25. The method of claim 23 wherein the cradle member is attached to the table assembly via a support arm rotatably extending from a connection interface to the cradle member.

26. The method of claim 22 wherein imaging the region of interest at a first angle comprises imaging a heart of the patient body.

27. The method of claim 22 wherein moving the patient platform to a first position comprises adjustably translating the patient platform away from the table assembly.

28. The method of claim 27 further comprising rotating an image detector away from the table assembly at the first angle.

29. The method of claim 22 wherein moving the patient platform to a second position comprises adjustably translating the patient platform towards the table assembly.

30. The method of claim 29 further comprising rotating an image detector towards the table assembly at the second angle.
